(19) United States
(12) Patent Application Publication
(10) Pub. No.: US 2012/0122795 A1
(43) Pub. Date: May 17, 2012

(54) ACCELERATED EXTENSION OF AXONS

(75) Inventors: Samantha Butler, Los Angeles, CA (US); Keith Phan, Los Angeles, CA (US)

(73) Assignee: University of Southern California

(21) Appl. No.: 13/205,513

(22) Filed: Aug. 8, 2011

Related U.S. Application Data
(60) Provisional application No. 61/401,261, filed on Aug. 9, 2010.

(51) Int. Cl. A61K 38/17; C12N 15/85; C12N 5/0797; A61K 31/7088; A61P 25/08; C12Q 1/02; C12Q 1/68; A61P 25/16; A61P 25/00; C12N 5/079; A61P 25/28

(52) U.S. Cl. 514/17.8; 435/375; 435/455; 514/17.7; 514/44 R; 435/366; 435/325; 435/29; 435/6.13

(57) ABSTRACT

The present disclosure relates to methods and compositions for promoting the extension of a neural cell by increasing the expression of activity of cofilin in the cell. The increase can be local to the end of the neural cell in need of extension and the neural cell can be a cell in an individual. By extending the neural cell to reach its synaptic target, the methods and compositions of the present disclosure can also be used for treating neurological diseases characterized by damaged or degenerated neurons.

### CROSS-REFERENCE TO RELATED APPLICATIONS

This application claims the benefit under 35 U.S.C. §119(e) of U.S. Provisional Application No. 61/401,261, filed on Aug. 9, 2010, the contents of which are hereby incorporated by reference in their entirety into the present disclosure.

### STATEMENT OF GOVERNMENT SUPPORT

This invention was made with government support under Grant No. R01 NS 063999 awarded by the National Institutes of Health. The government has certain rights in the invention.

### FIELD OF TILE INVENTION

The present technology relates generally to methods and compositions for extending neural axons and treatment of neurological diseases.

### BACKGROUND

Throughout the disclosure, various technical and patent literatures are identified by the authors' names and year of publication. The full bibliographical citations of these literatures can be found in the text of this specification or immediately preceding the claims. The content of these disclosures are incorporated into the present specification by reference in their entirety.

After injury or a degenerative disease, it is difficult to connect the neural cells in the central nerve system (CNS) back to their synaptic targets. The difficulties arise from (a) the inhibitory environment of the CNS and (b) the distances that the neuronal processes, or axons, have to grow to regenerate a neural circuit. For example, it takes years for axons to reinnervate an adult leg to recover movement after a spinal cord injury.

During development, axons extend along stereotyped pathways to form precisely ordered neuronal networks. Axons are guided along these pathways by directional information in the embryonic environment, which instructs the axonal growth cones to locally reorganize the actin cytoskeleton and thereby move towards or away from the source of the signal. Additionally, axons must reach directional information at the right time during development and then modulate their speed to navigate the guidance signal. What controls this process is unknown.

The trajectory of dorsal commissural axons around the circumference of the developing spinal cord is a key model system for elucidating axon guidance mechanisms. Commissural neurons (dI1s) arise adjacent to the dorsal midline and are generated by the Bone Morphogenetic Proteins (BMPs) in the roof plate (RP). The BMPs subsequently act as a guidance cue to orient commissural axons ventrally. The ability of inductive growth factors, such as the BMPs, to direct remarkably disparate cellular processes during neural development has been observed for other morphogens. However, the mechanisms by which morphogens achieve these distinct functions remains largely unresolved.

### SUMMARY

It is discovered that there exists a balance between the activation states of cofilin and Limk1 that regulates the rate of axon outgrowth by regulating how fast actin treadmills in the growth cone. The inventors demonstrate that elevation of cofilin activity, either directly or by reducing Limk1 activation in a BMP-dependent manner, results in faster axons growth. Moreover, upregulation of cofilin also results in larger and more complex growth cones. Therefore, the inventors have identified a cell-intrinsic way in which axon outgrowth can be accelerated in vivo. Alteration of this intrinsic pathway can then be used to extend axons in vivo and the extension can be location specific. Therefore, a neural stem cell or neural precursor cell can be extended to provide a valuable source for therapy.

Thus, one embodiment of the present disclosure provides a method for promoting extension of a neural cell, comprising, or alternatively consisting essentially of, or yet alternatively consisting of, increasing the biological activity of cofilin in the cell.

In one embodiment, the increasing of the biological activity of cofilin in the cell comprises introducing into the cell an isolated or recombinant cofilin polypeptide or an isolated or recombinant polynucleotide encoding the polypeptide. The isolated or recombinant cofilin polypeptide, in some aspects, comprises a mutation that inhibits phosphorylation of the cofilin polypeptide. In another aspect, the isolated or recombinant mutant cofilin polypeptide comprises, or alternatively consists essentially of, or yet further consists of, the amino acid sequence of SEQ ID NO: 1 or a biological equivalent thereof.

In another embodiment, increasing of the biological activity of cofilin in the cell comprises inhibiting the expression of the biological activity of Limk1 in the cell. The inhibiting of the activity of Limk1 in the cell, in some aspects, comprises introducing into the cell an isolated or recombinant Limk1 polypeptide mutant that does not phosphorylate cofilin or an isolated or recombinant polynucleotide encoding the polypeptide mutant. In another aspect, the Limk1 mutant does not have one or more of LIM or PDZ domain see Meng et al. Neuron 35(1):121-33, 2002).

In yet another embodiment, the increasing of the biological activity of cofilin in the cell comprises, or alternatively consists essentially of, or yet further consists of, inhibiting the expression of, or the biological activity of BmprII. The inhibiting of the activity of BmprII, in some aspects, comprises or alternatively consists essentially of, or yet further consists of introducing into the cell an isolated or recombinant BmprII polynucleotide mutant that does not phosphorylate Limk1 or a polynucleotide encoding the BmprII polynucleotide mutant. In another aspect, the BmprII polynucleotide mutant comprises, or alternatively consists of, the amino acid sequence of SEQ ID NO: 2 or a biological equivalent thereof.

In some embodiments, the biological activity of cofilin in the neural cell is increased at a location in the cell proximate to an end of the cell in need of extension. In some aspects, the neural cell comprises a commissural axon or a motor axon.

In some embodiments, the neural cell is a neural stem cell or a neural precursor cell. The neural stem cell can be derived from an induced pluripotent stem cell (iPSC), an embryonic stem cell or a parthenozenetic stem cell.

In some embodiments, the neural cell is a damaged or degenerated neuro cell that is terminally differentiated.

Yet still in some embodiments, the increasing of the biological activity of cofilin is in vivo or ex vivo. In one embodiment, the neural cell is a human neural cell.

Also provided, in one embodiment, is an extended neural cell prepared by any of the above methods, and compositions comprising or alternatively consists essentially of, or yet further consists of the cell and a carrier. Further provided, in one embodiment, is an amino acid sequence comprising or alternatively consists essentially of, or yet further consists of, the sequences of SEQ ID NO: 1 or 2, or a nucleic acid sequence encoding the amino acid sequence, or a composition of the amino acid sequence or nucleic acid sequence and a carrier.

The present disclosure further provides, in one embodiment, a neural cell comprising, or alternatively consisting essentially of, or yet alternatively consisting of, an isolated or recombinant polypeptide comprising or alternatively consists essentially of, or yet further consists of, an amino acid sequence of SEQ ID NO: 1 or 2 or a biological equivalent thereof, or an isolated or recombinant polynucleotide comprising or alternatively consists essentially of, or yet further consists of a nucleic acid sequence encoding SEQ ID NO: 1 or 2 or a biological equivalent thereof.

In one embodiment, the polypeptide or polynucleotide is localized at a location in the cell proximate to an end of the cell in need of extension.

In another embodiment, the neural cell is a neural stem cell or a neural precursor cell. The neural stem cell can be derived from an induced pluripotent stem cell (iPSC), an embryonic stem cell or a parthenogenetic stem cell.

In another embodiment, the neural cell is a damaged or degenerated neural cell that is terminally differentiated.

Further provided, in one embodiment, is a population of any of the above neural cells. In one aspect, the population is a substantially homogenous population of any of the above cells, wherein the homogeneous population is comprises at least 50% or more of the neural cells,

In one embodiment, the present disclosure provides a method for treating a neurological disease characterized by a damaged or a degenerated neural cell, comprising, or alternatively consisting essentially of, or yet alternatively consisting of, increasing the biological activity of cofilin in the neural cell to promote the extension of the neural cell, thereby treating the disease.

In one embodiment, the present disclosure provides a method for treating a neurological disease characterized by a damaged or a degenerated neural cell, comprising, or alternatively consisting essentially of, or yet alternatively consisting of, introducing into the neural cell an isolated or recombinant polypeptide comprising or alternatively consists essentially of or yet further consists of an amino acid sequence of SEQ ID NO: 1 or 2 or a biological equivalent thereof, or an isolated or recombinant polynucleotide comprising or alternatively consists essentially of, or yet further consists of a nucleic acid sequence encoding SEQ ID NO: 1 or 2 or a biological equivalent thereof.

In one embodiment, the present disclosure provides a method for treating a neurological disease characterized by a damaged or a degenerated neural cell in a subject, comprising, or alternatively consisting essentially of, or yet alternatively consisting of, implanting to the subject any of the above neural cells or cell population.

A neurological disease characterized by a damaged or a degenerated neural cell includes, for example without limitation, Traumatic Brain injury, Alzheimer's disease, Parkinson's disease, epilepsy, Huntington's disease or stroke.

In one embodiment, a method is provided for identifying an agent suitable for increasing the biological activity of cofilin, comprising, or alternatively consisting essentially of, or yet alternatively consisting of, contacting a candidate agent with a neural cell, wherein increased extension of the neural cell and an increased phosphorylation of cofilin as compared a neural not in contact with the agent indicates that the agent is suitable for increasing the biological activity of cofilin.

Also provided are kits for use in promoting extension of a neural cell or treating a neurological disease characterized by a damaged or a degenerated neural cell, comprising, or alternatively consisting essentially of or yet alternatively consisting of, an isolated or recombinant polypeptide comprising or alternatively consists essentially of, or yet further consists of an amino acid sequence of SEQ ID NO: 1 or 2 or a biological equivalent thereof, or an isolated or recombinant polynucleotide comprising or alternatively consists essentially of, or yet further consists of a nucleic acid sequence encoding SEQ ID NO: 1 or 2 or a biological equivalent thereof and instructions to use.

Still further provided, in one embodiment, is a kit for use in promoting extension of a neural cell or treating a neurological disease characterized by a damaged or a degenerated neural cell, comprising, or alternatively consisting essentially of or yet alternatively consisting of, any neural cell or cell population of the above embodiments and instructions to use.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A-C are pictures showing distribution of Limk1 and phosphorylated (p) cofilin in the developing spinal cord.

(A) Limk1 is broadly expressed in post-mitotic neurons in the E10.5 mouse spinal cord.

(B, C) Limk1 protein (red) present in the processes of many post-mitotic spinal neurons, including the Tag1⁺ (green) commissural axons in the dorsal spinal cord (C).

(D, E) In dissociated commissural neurons from E13 rat embryos. Limk1 protein is present throughout the cell body, axon and growth cone (arrowhead. D), whereas BmprII, the binding partner of Limk1, is present primarily in the commissural growth cone (arrowhead, E).

(F-G) Antibodies against p-cofilin (red) also label many post-mitotic neurons and their processes (open arrowheads, F) in the dorsal and intermediate spinal cord including the Tag1⁺ (green) commissural neurons. The area boxed in (F) is shown at higher magnification in (G). Scale bar: (A-C, F) 40 μm (D, E) 10 μm (G) 12 μm.

FIG. 2A-H show that cofilin is acutely phosphorylated following stimulation with BMP7.

(A-H) Cultures of dissociated E13 rat commissural neurons were briefly stimulated with either water or BMP7 recombinant protein and then labeled with antibodies against p-cofilin (green, A-D), total cofilin (green, E, F), and neuronal class III β-Tubulin (Tuj1, A, C, E, F) to reveal neuronal processes (Geisert and Frankfurter. 1989) or subjected to a Western analysis (H).

(A, B) In unstimulated culture conditions, p-cofilin is evenly distributed at low levels across commissural growth cones.

(C, D) In contrast, the level of p-cofilin was dramatically increased in commissural growth cones treated with BMP7, suggesting that the inactivation of cofilin in commissural growth cones is an immediate consequence of BMP7 treatment. Panels A-D were imaged using identical settings on the confocal microscope, whereas panels C′ and D′ were imaged at a lower gain/offset levels to reveal the distribution of p-cofilin in the commissural growth cone after stimulation. In this example, p-cofilin is most highly upregulated in the transition zone of the growth cone (open arrowhead, D′) where actin-bundles are thought to be severed.

(E, F) The distribution and levels of total cofilin protein is similar in unstimulated (E) and BMP7-treated (F) commissural growth cones, suggesting that BMP7 treatment directly results in the phosphorylation of cofilin rather than a redistribution of cofilin protein.

(G) Quantification of mean p-cofilin immunofluorescence in stimulated and unstimulated commissural growth cones. There was a significant increase (p<2.1×10⁻⁹, student's t-test) in the level of p-cofilin after addition of BMP7 (n=162 neurons') compared to unstimulated control cultures (n=117 neurons).

(H) Western blot of dissociated E13 neurons stimulated with either water or BMP7. There was significant increase (p<0.0023) in the level of p-cofilin following BMP-stimulation (n=4 experiments).

Scale bar: (A-F) 10 μm.

FIG. 3A-I show that constitutive activation of Limk1 results in commissural axon outgrowth defects

(A, B) Chick commissural neurons electroporated with farnesylated (f) GFP under the control of the Math1 enhancer (Math1::fGFP) at Hamburger Hamilton stage (HH) 15, extend GFP⁺ axons (red) to the floor plate (FP, arrowhead, B) by HH stage 24 in concert with the non-electroporated Axonin1⁺ axons (green).

(C-E). In contrast, chick, commissural neurons expressing both a myc-tagged truncated form of Limk1 (caLimk1-myc, red) and GFP have dramatically reduced axon outgrowth at RH stage 24 (arrowhead, D). caLimk1-myc (k1) is constitutively active, it lacks the Lim domain and thus is neither auto-inhibited nor binds to BmprII. A higher magnification image (E) revealed that the Myc⁺ GFP⁺ commissural growth cones stall immediately adjacent to the commissural cell bodies (arrowheads, D, E) suggesting that activation of cofilin has been repressed to such an extent that actin dynamics have been frozen in the commissural growth cone.

(F, C) Consistent with this model, this outgrowth phenotype can be partially rescued by concomitantly electroporating commissural neurons with a non-phosphorylatable form of cofilin, cofilinS3A-his (green). Panels F′ and G′ are higher magnification views of the electroporated region of the spinal cords in F and G. Only His⁺ (green) or Myc⁺ H⁺ is (yellow) neurons extend axons into the intermediate and ventral spinal cord (arrows, G), very limited axon outgrowth was observed for the Myc⁺ (red) neurons.

(H) The extent of the commissural axon outgrowth was quantified by determining whether commissural axons had crossed one of four arbitrary lines in the spinal cord: mid-dorsal (MD), intermediate (INT), mid-ventral (MV) or the FP. Given the potential variation between experiments, only embryos electroporated within the same experiment were compared to each other.

(I) Although 63.7%±8.2 of commissural neurons expressing Math1::caLimk-myc (n=24 sections from 3 embryos) extended a neurite by HH stage 23, only 8% reached the INT line (p<5.8×10⁻¹² different from Math1:fGFP control, student's t-test) and none extended to the MV line) (p<2.6×10⁻¹⁰). In contrast, 86.7%±2.9 of commissural neurons co-expressing Math1::caLimk-myc and Math1::cofilinS3A-his (n=108 sections from 6 embryos) extend axons of which 47% reach the INT line (p<1.1×10⁻¹² different from Math1::caLimk-myc alone) and 17% reach the MV line (p<9.8×10⁻⁶).

Scale bar: (A-D, F, G) 40 μm (E, F′, G′) 20 μm

FIG. 4A-C show that commissural axons have accelerated trajectories in Limk1 mutant mouse spinal cords.

(A-F) The extent of commissural axon outgrowth was assessed in stage E10.5 wild type and Link1^{-/-} embryos. Commissural axons were detected using a genetically encoded reporter, Math1::tauGFP (green, A-F). Sections were also labeled with antibodies against Lhx2/9 (red, A, D) to normalize the stage of commissural neuron development between sections.

(A-C). In sections of E10.5 wild-type spinal cord with between 11-20 Lhx2/9⁺ cells on each side Math1⁺ axons are in the process of projecting through the intermediate spinal cord (arrowhead, A, B) with relatively few Math1⁺ axons having reached the FP. The FP is boxed in (A) and is shown at higher magnification in (C).

(D-F). In contrast, in Limk1^{-/-} littermates at the same stage of commissural neural development, more axons have projected thought the intermediate spinal cord (arrowhead, D, E) and have reached the FP. The FP is boxed in (D) and is shown at higher magnification in (F).

(G). Quantification of the total number of axons in the FP at three stages of commissural neuronal development: 1-10 Lhx2/9 neurons (wild-type: n=133 axons from 16 embryos, Limk1^{-/-}: n=109 axons from 10 embryos), 11-20 Lhx2/9⁺ neurons (wild-type: n=99 axons from 15 embryos, Limk1^{-/-}: n=51 axons from 7 embryos) and 21-30 Lhx2/9⁴ neurons (wild-type: n=47 axons from 7 embryos, Limk1^{-/-}: n=22 axons from 4 embryos). For the two earliest stages of development, there are 20-25% more Math1⁺ axons in the FP in Limk1^{-/-} mutants compared to wild-type littermates (1-10 neurons, p<0.0038, student's t-test, 11-20 neurons p<0.0021). At later stages there is no significant difference (p>0.34) in the numbers of axons crossing the FP in wild-type and Limk1^{-/-} embryos. However, the tract becomes increasingly fasciculated in the FP at later stages making it challenging to distinguish individual axon trajectories.

Scale bar: (A, B, D, E) 40 μm (C, F) 5 μm.

FIG. 5A-K show that reducing Limk1 activity by truncating BmprII results in accelerated axon growth.

(A-K) To assess whether Limk1 functionally interacts with BmprII in commissural neurons, a truncated BmprII construct was generated under the control of the Math1 enhancer (green, Math1:: BmprIIΔLim-GFP) in which the Limk1 binding site on the intracellular tail of BmprII was replaced with GFP. Commissural neurons were labeled with antibodies against Lhx2/9 (red, A, C) or Axonin1 (red, E, G). Commissural neurons electroporated with full-length BmprII project axons in a similar manner to control GFP⁺ axons (data not shown).

(A, B, E, F) Chick neurons electroporated with a control Math1:fGFP construct at HH stage 15, have extended GFP⁺ axons into the intermediate spinal cord by HH stage 20.

(C, D, C, H) In contrast, many commissural neurons electroporated with Math1.::BmprIIΔLim-GFP have extended axons to the FP (arrowhead, D, H) by HH stage 20.

(I) Axon outgrowth was quantified as follows:

HH stage 19: 95%±4.6 commissural neurons expressing Math1::fGFP extended axons, of which 10% had projected to the INT line (n 90 sections from 5 embryos). A similar number, 86%±3.0 (p>0.05, student's t-test) of commissural neurons expressing Math1::BmprIIΔLim-GFP sections had extended axons, however about 30% (p<1.4×10⁻⁷) of these axons had reached INT line (n=81 sections from 5 embryos) HH stage 20: 88%±5.1 of commissural neurons expressing Math1::fGFP extended axons, with 4.5% of these axons projecting to the MV line (n=52 sections from 4 embryos). A similar number, 85%±5.1 (p>0.33) of commissural neurons expressing Math1::BmprIIΔLim-GFP have extended axons, however over 32% (p<2.6×10⁻¹¹) of these had reached MV line (n=51 sections from 4 embryos).

HH stage 21. 75.3%±2.2 of commissural neurons expressing Math1::fGFP extended axons, with 12% of these axons projecting to the FP (n=74 sections from 5 embryos). In this experiment, 88.6%±3.0 of commissural neurons expressing Math1::BmprIIΔLim-GFP had extended axons, of however over 26% (p<1.4×10⁻¹³) of these had reached FP (n=42 sections from 3 embryos).

(J) To assess whether endogenous BMP binding was required for accelerated axon growth, a construct lacking both the extracellular domain (E) and Lim binding domain (Math 1:: BmprIIΔEΔLim-GFP) was electroporated into commissural neurons. By HH stage 21 there was no significant difference between extent of outgrowth of control GFP⁺ axons (n=114 sections from 7 embryos) compared to BmprIIΔEΔLim-GFP⁺ axons (n=97 sections from 5 embryos) at either the MV (p>0.057) or FP (p>0.066) lines.

(K) The rate of growth of electroporated axons was directly determined by imaging live cultures. Axons electroporated with Math1: have a velocity of 13.4±1.1 μm/hr (n=28 neurons), in contrast axons electroporated with Math1::BmprIIΔLim-GFP grow significantly faster (p<0.018) with a velocity of 17.6±1.3 μm/hr (n=31 neurons).

Scale bar: (A-H) 40 μm.

FIG. 6A-G demonstrate that Accelerated commissural neurons have more complex growth cones.

(A, B) Commissural neurons electroporated either with Math1::BmprIIΔLim-GFP (data not shown) or with Math1::cotilin-myc (B, B′) extend growth cones in vivo that appear to have longer, more extensive filopodia that control growth cones (A, A′).

(C-F) To assess the morphology of electroporated commissural neurons in vitro, chick embryos were electroporated at HH stage 15 and cultures of dissociated dorsal neurons were generated at HH stage 24. Control (A, B) and BmprIIΔLim-GFP⁺ (C, D) commissural growth cones were labeled with antibodies against class III β-tubulin (Ttuj1, blue) and the Erm (ezrin, radixin, moesin) complex (red) to reveal the growth cone morphology. The BmprIIΔLim-GFP⁺ growth cones are dramatically more complex, with longer and more extensive filopodia.

(E) Quantification revealed that although there was no significant difference between control and experimental neurons in the length of the longest neurite (p>0.13, student's t-test), the average perimeter of the BmprIIΔLim-GFP⁺ growth cones (150.4 μm+2.0, n=41) is over 50% longer (p<0.015) than that of the controls (90.9 μm±1.1, n=50). (G) shows no difference in overall neurite length was observed in vitro.

Scale bar: 10 μm.

FIG. 7A-F present images and charts to show that accelerated commissural axons make guidance errors projecting towards and across the floor plate.

(A-D) To determine the consequence of accelerated axon growth, the behavior of electroporated commissural axons crossing the FP was examined. Chick embryos were electroporated at HH stage 15 and longitudinal open book preparations of the spinal cord were generated at HH stage 24. The position of the FP was visualized by labeling the flanking motor neuron column with antibodies against Islet1/2 (Isl1/2, blue, F, H). The images in A-C are flattened confocal stacks, whereas the image in D is a single confocal slice to facilitate visualizing axons turning rostrocaudally after crossing the FP.

(A, B) By HH stage 24, control neurons electroporated with Math1::fGFP have projected axons ventrally and then sharply rostrally, to project towards the brain. There are two classes of GFP⁺ axons, an ipsilateral population that turns before the FP (bracket A, arrow B) and a contralateral commissural population that turns after crossing the FP (bracket A, arrowhead B).

(C, D) In contrast, by the same stage, neurons electroporated with Math1::BmprIIΔLim-GFP project very few axons ipsilaterally (dotted bracket, C) and the contralaterally projecting commissural axons turn both rostrally and caudally (arrowheads, C, D).

(E) Whereas 18.4%±1.9 (n=1137 axons from 4 open book preparations) of control GFP⁺ axons turn ipsilaterally in the intermediate spinal cord, only 2.9%±0.88 of BmprIIΔLim-GFP⁺ axons (n=1341 axons from 9 open book preparations) make the ipsilateral turn (p<1.3×10⁻⁷ significantly different from control, student's t-test). Similarly, only 6.3%±0.88 of axons overexpressing cofilin-myc (1286 axons from 6 open book preparations) make the ipsilateral turn (p<3.0×10⁻³ significantly different from control, p>0.08 different from BmprIIΔLim-GFP⁺ axons).

(F) The number of axons in the FP that have turned either rostally or caudally by HH stage 24 were determined. The majority of control commissural axons turn rostrally (52.1%±1.4 of axons have turned rostrally, 1.4%±0.3 of axons turn caudally, n=814 axons from 4 open book preparations) whereas, significantly more of the axons electroporated with either Math1::BmprIIΔLim-GFP (48.1%±3.9 of axons turn rostrally, 14.6%±2.9 of axons turn caudally, n=1931 axons from 10 open book preparations, p<1.5×10⁻⁶ different from control) or Math1::cofilin-myc (51.9%±3.6 of axons turn rostrally, 10.4%±0.8 of axons turn caudally, n=1045 axons from 6 open book preparations, p<1.9×10⁻⁶ different from control) turn caudally.

Scale bar: (A, C) 30 μm (B, D) 20 μm.

FIG. 8A-D show that truncating the Limk1-binding domain of BmprII results in an increase in cofilin activity.

(A) Model by which BMP binding to the Bmpr complex activates Limk1 and thereby inactivates cofilin.

(B) Model by which BmprIIΔLim-GFP results in the increased activation of cofilin. Eectroporating the truncated BmprII construct into chick embryos will result in two forms of BmprII being present in commissural neurons: the endogenous BmprII and the deletion construct. Some of the time, the truncated BmprII will sequester the endogenous type I Bmprs leaving Limk1, bound to the endogenous BmprII, “primed” but inactive, thereby resulting in an increase in cofilin activity.

(C) Western blot, sequentially probed with antibodies against p-cofilin and actin, of BMP7-stimulated (lane A) untransfected COS cells, (lane B) COS cells transfected with the wild-type Bmpr complex and Limk1/cofilin alone, and in combination with (lane C)× or (lane D) 2× BmprIIΔLim-GFP. α-pcofilin detects both endogenous and transfected cofilin protein. The amount of DNA was standardized between the different transfections.

(D) To quantify the endogenous p-cofilin protein levels, the pixel intensity of the pcofilin bands in three independent Western analyses were normalized against the respective actin loading control bands and plotted relative to the levels of p-cofilin in the untransfected control (lane A). This quantification demonstrated that whereas the transfection of wild-type BmprII protein results in an increase in p-cofilin, the transfection of BmprIIΔLim-GFP results in a significant decrease (p<0.04, student's test) in p-cofilin protein, suggesting that cofilin is now more active.

FIG. 9A-B show that phosphorylated cofilin levels are reduced in Limk1 mutant spinal cords.

(A) Western blot of spinal cords dissected from a wild-type or Limk1^{-/-} E11.5 embryo probed with antibodies against either p-cofilin or actin.

(B) There was significant decrease (p<0.019) in the level of p-cofilin in the Limk1^{-/-} animals compared to wild-type littermates (n=3 Lunk1^{-/-} embryos, 5 Limk1^{-/-} embryos taken from 3 litters).

FIG. 10A-I show that the Math1 enhancer directs comparable levels of expression of BmprIIΔLim-GFP and BmprIIΔEΔLim-GFP. Panels (A-I) show commissural neurons were electroporated with either fGFP (A, B) or BmprIIΔLim-GFP (D, E) or BmprIIΔEΔLim-GFP (G, H) under the control of the Math1 enhancer at stage 15. The levels of electroporated protein were assessed at HH stage 22 using antibodies against GFP (green, A, B, D, E, G, H) and Lhx2/9 (red, A, D, G). Panels A, B, D, E, G and H were imaged using identical settings on the confocal microscope. The intensity of GFP⁺ fluorescence (C, F, I) was measured along the yellow dotted line in panels A, D and C. The extent of GFP⁺ fluorescence observed was comparable for the three electroporation conditions (GFP: 3584±88 arbitrary units, n=51 samples from 5 sections; BmprIIΔLim-GFP: 3455±86 arbitrary units, n=61 samples from 7 sections; BmprIIΔEΔLim-GFP: 3635±88 arbitrary units, n=63 samples from 5 sections) suggesting that the Math1 enhancer directs similar levels of gene expression (p>0.12, student's t-test that the expression levels of the GFP fusion proteins are similar).

Scale bar: 4 μm.

FIG. 11A-I show that truncating the Limk1-binding domain of BmprII has no effect on the activity of Smad1/5/8.

(A-I) Commissural neurons were electroporated with either fGFP (green, A, B) or BmprIIΔLim-GFP (green, D, E) or BmprIIΔEΔLim-GFP (green, G, H) under the control of the Math1 enhancer at HH stage 15. Smad1/5/8 activity was examined at HH stage 22 using antibodies against the phosphorylated form of Smad1/5/8 (red, A, B, D, E, G, H). The level of Smad⁺ and GFP⁺ fluorescence intensities (C, F, I) were measured along the yellow dotted line in panels A, D and G. In all three cases, the extent of Smad⁺ fluorescence on the electroporated side was comparable to that on the non-electroporated side. Most critically, there was no alteration in the levels of Smad1/5/8 phosphorylation in cells also expressing GFP as was observed after the electroporation of constitutively active forms of the type I Bmpr (Yamauchi et al 2008).

Scale bar: 25 μm.

FIG. 12A-Q show that electroporation of Math1::BmprIIΔLim-GFP does not result in dorsal cell fate changes.

(A-P) Commissural (dI1) neurons were electroporated with either fGFP (green, A, B, E, F, I, J, M, N) or BmprIIΔLim-GFP (green, C, C, H, K, L, O, P) under the control of the Math1 enhancer at HH stage 15. Changes in cellular identity were examined using antibodies against either Lhx2/9+ (panLh2, red, A-H) or Lhx9+ (red, I-P) at HH stage 18 (A-D), when the Math1+ neurons first extend axons, HH stage 20/21 (E-L), when Math1+ axons have reached the intermediate spinal cord, and HH stage 24 (M-P) when the commissural axons have extended across the FP and the ipsilaterally projecting axons have begun to turn rostrally. At these stages, all of the Math1+ neurons are Lhx94+ (inset, J). No alterations either in the number of Lhx9+ neurons or the timing of their development were observed. However, axon outgrowth was consistently more advanced in the HH stage 20/21 embryos electroporated with Math1::BmprIIΔLim-GFP (arrowhead, G) compared to controls (arrowhead, E).

(Q) Quantification of the number of Lhx2/9+ neurons on the electroporated side verses the non-electroporated side following electroporation of either Math1::fGFP or Math1::BmprIIΔLim-GMP. For both fGFP and BmprIIΔLim-GFP, there was no change in the number of Lhx2/9+ neurons either at HH stage 18 (fGFP: p>0.23, student's t-test, n=22 sections from 3 embryos; BmprIIΔLim-GFP: p>0.30, n=34 sections from 2 embryos) or at HH stage 20 (fGFP: p>0.30, n=53 sections from 4 embryos; BmprIIΔLim-GFP: p>0.47, n=65 sections from 4 embryos) Similarly, there was no significant difference between the number of Lhx2/9+ neurons on the electroporated sides of fGFP+ and BmprIIΔLim-GFP+ embryos at either HH stage 18 (p<0.35) or HH stage 20 (p<0.09).

Scale bar: 40 μm.

FIG. 13A-G show that overexpression of cofilin results in accelerated commissural axon outgrowth.

(A-F) Commissural neurons were electroporated at stage HH stage 14 and the extent of commissural axon outgrowth assessed at HH stage 19/20 using antibodies against GFP (green) and Lhx2/9 (red).

(A-C) By this developmental stage, commissural neurons electroporated with Math1::fGFP have extended GFP+ axons into the intermediate spinal cord (arrowhead, C).

(D-F) In contrast, commissural neurons electroporated with a myc-tagged form of cofilin under the control of the Math1 promoter have extended axons to the FP (arrowhead, H).

(G) Axon outgrowth was quantified as described in FIG. 3H. The quantification of axon outgrowth from BmprIIΔLim-GFP+ neurons at HH stage 19, also shown in FIG. 5I, is included here for comparison purposes. 111%±7.0 commissural neurons expressing Math1::fGFP extended axons, of which 10% had projected to the INT line (n=60 sections from 6 embryos). A similar number, 97%±4.4 (p>0.14, student's t-test) of commissural neurons expressing Math1L::cofilin-myc had extended axons, however significantly more, over 40% (p<18×10₋₈), of these axons had reached INT line (n=59 sections from 5 embryos).

Seale bar: 40 μm.

FIG. 14A-H show that misexpression of BmprIIΔLim-YFP in ventral and intermediate spinal neurons results in their extending complex growth cones.

(A-G) Chick embryos were electroporated at HH stage 15 with either CMV: YFP or CMV::BmprIIΔLim-YFP and cultures of dissociated ventral and intermediate neurons were generated at HH stage 24. Control (A-C) and BmprIIΔLim-YFP (D-G) growth cones labeled with antibodies against class III ®tibulin (Tuj1, blue) and the Firm (ezrin, radixin, moesin, red) complex to reveal the growth cone morphology. Similar to what was seen for the preparations of dorsal spinal neurons, the BmprIIΔLim-YFP growth cones are more complex than the controls, with longer and more extensive filopodia.

(H) Quantification revealed that although there was no significant difference between control and experimental neurons in the length of the longest neurite (p>0.39, student's nest), the average perimeter of the BmprIIΔLim-YFP growth cones (23.8 μm±1.7, n=33) is almost 50% longer (p<2.2×10⁻⁵, student's t-test) than the perimeter of control growth cones 2.6 μm±1.7, n=24).

Scale bar: 7 μm.

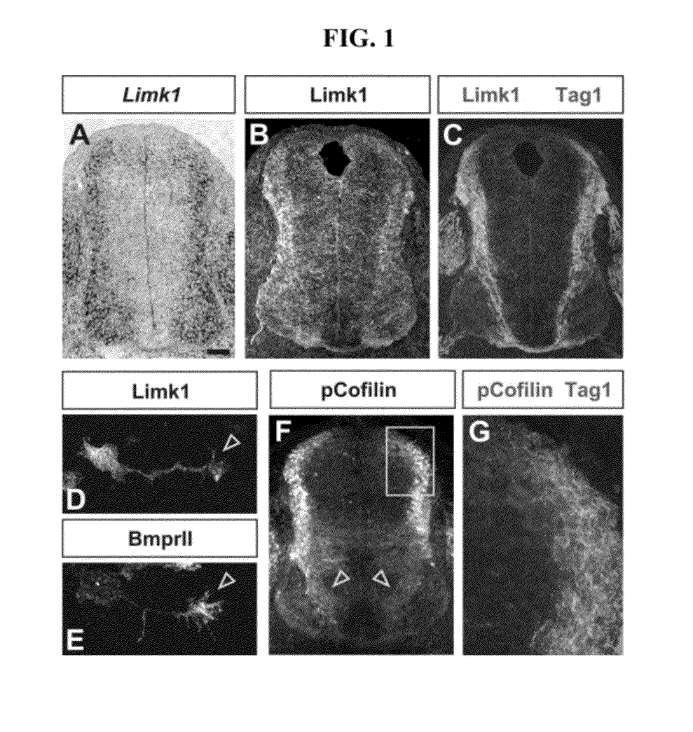

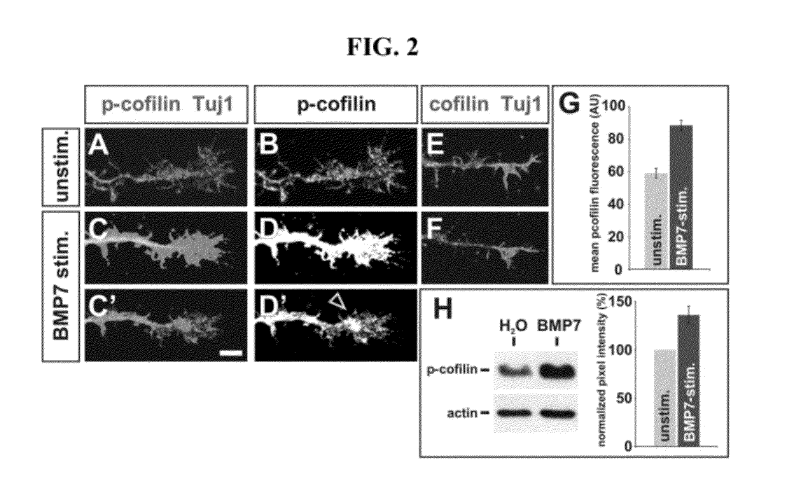

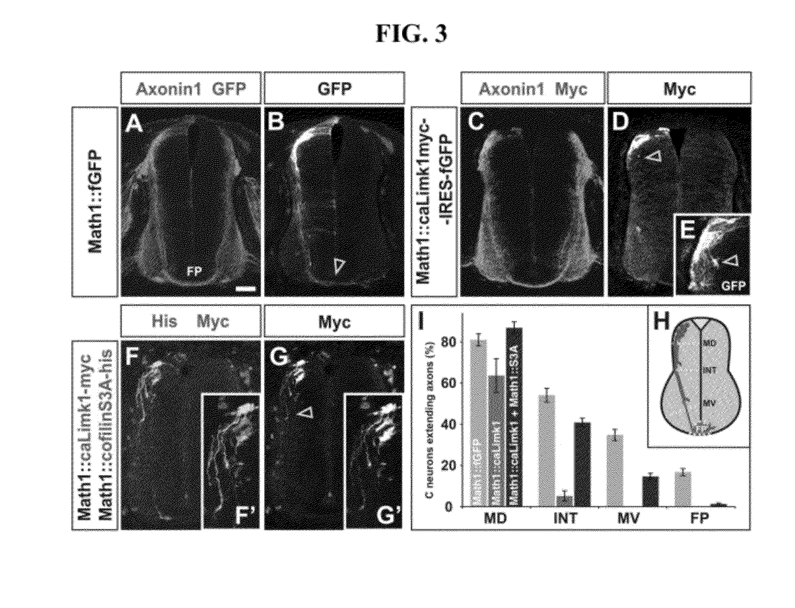

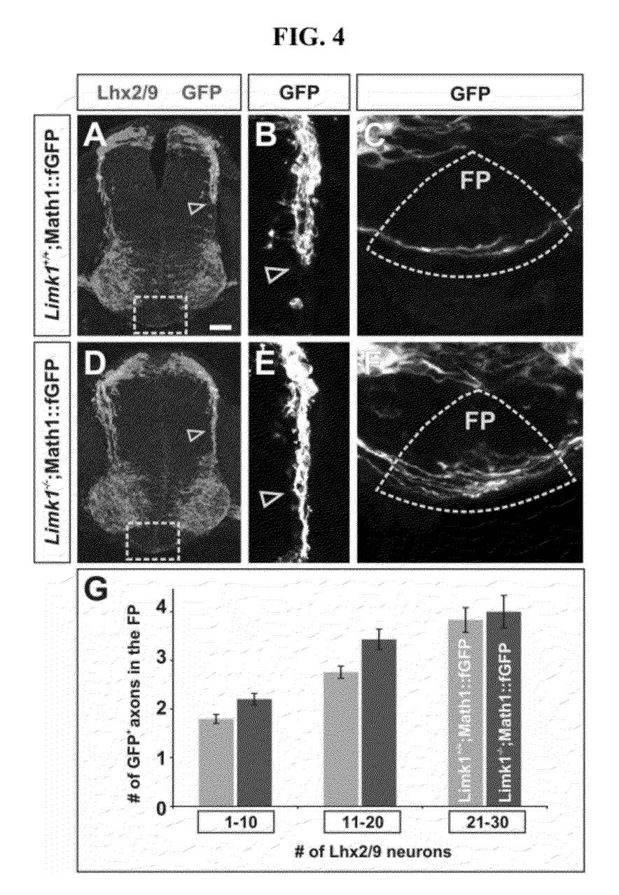

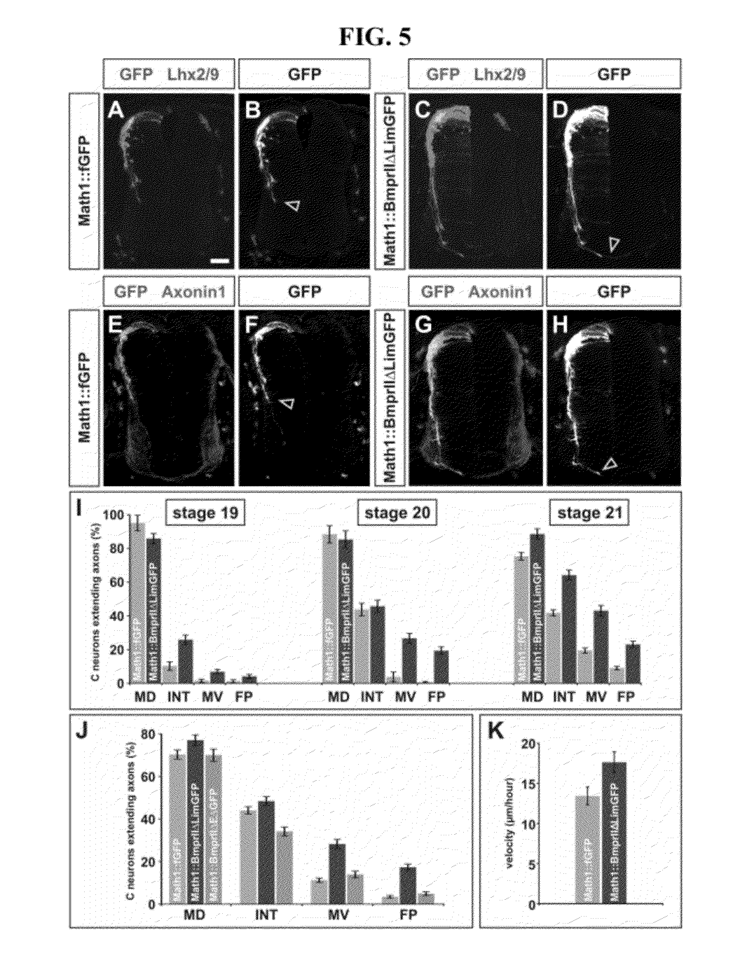

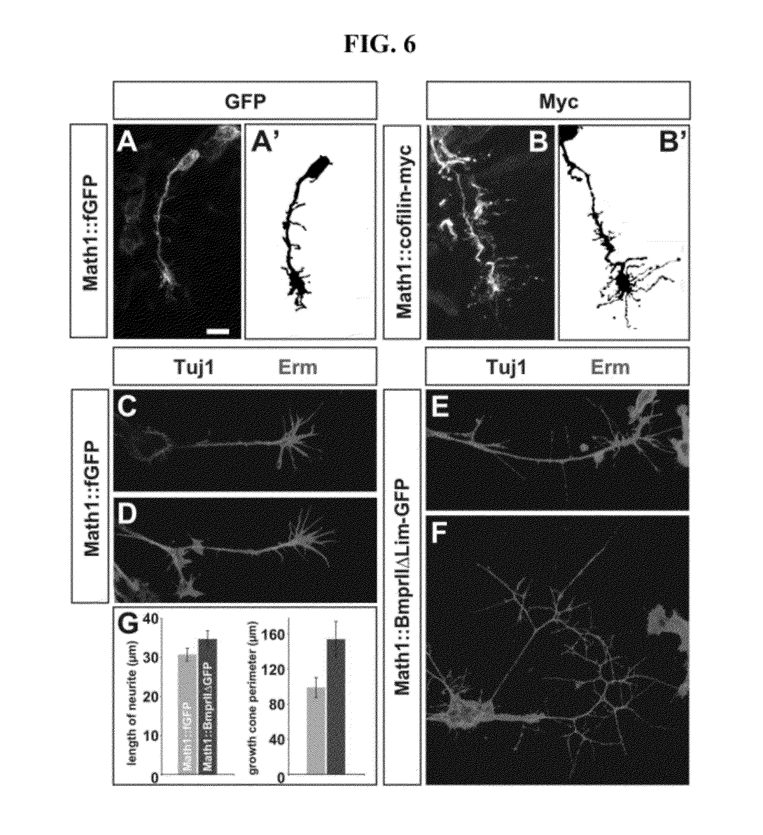

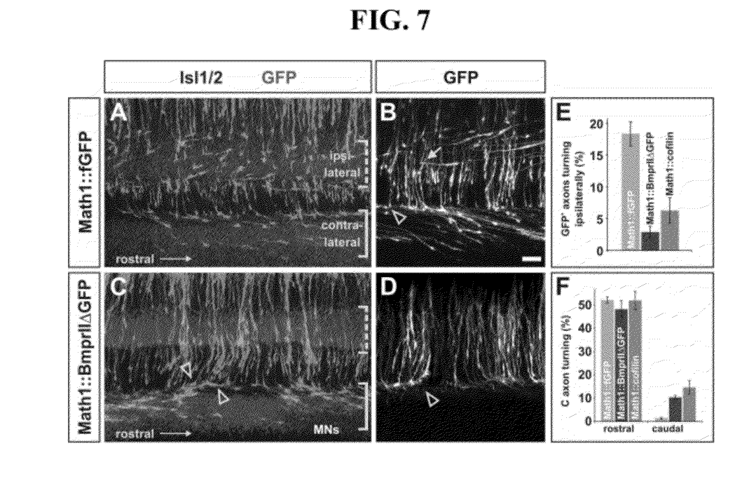

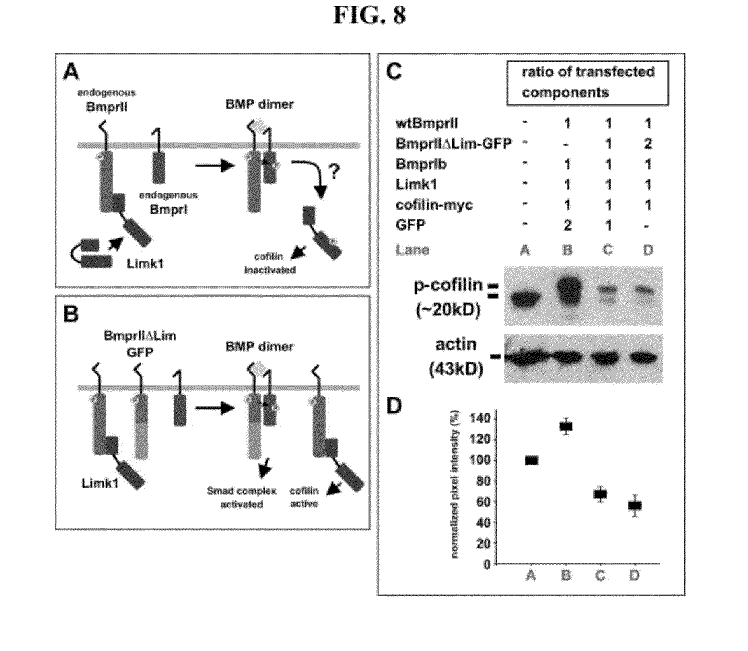

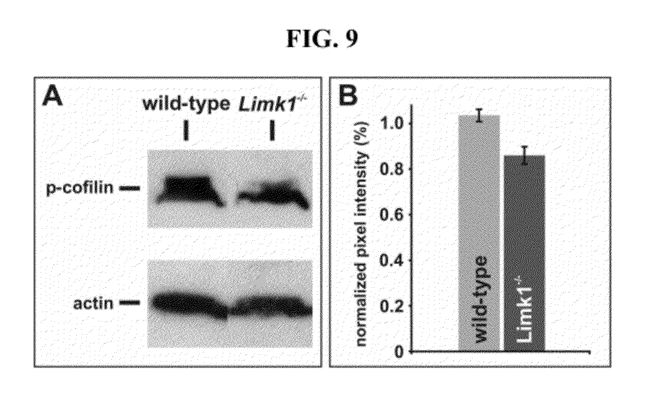

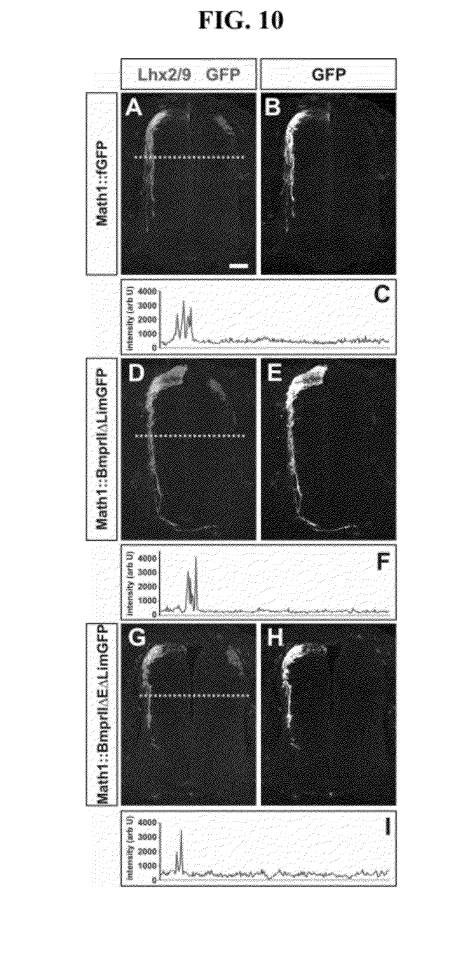

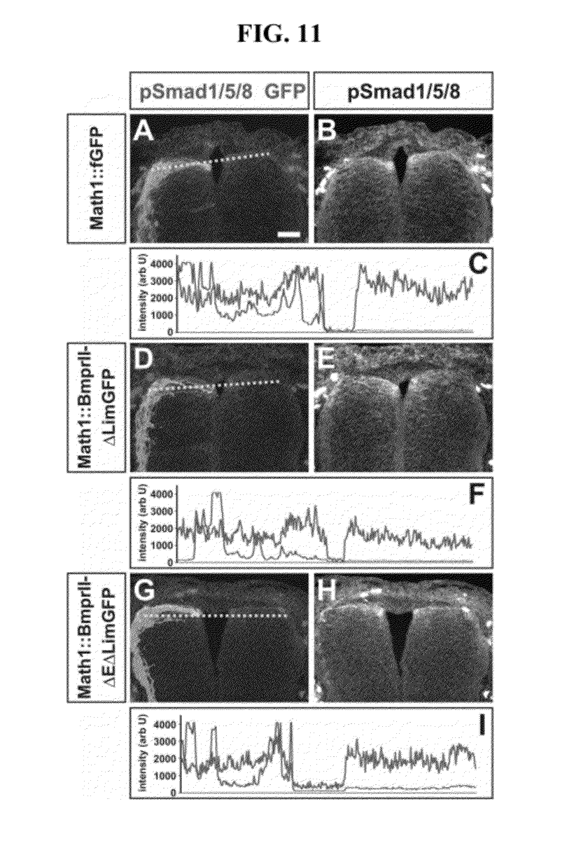

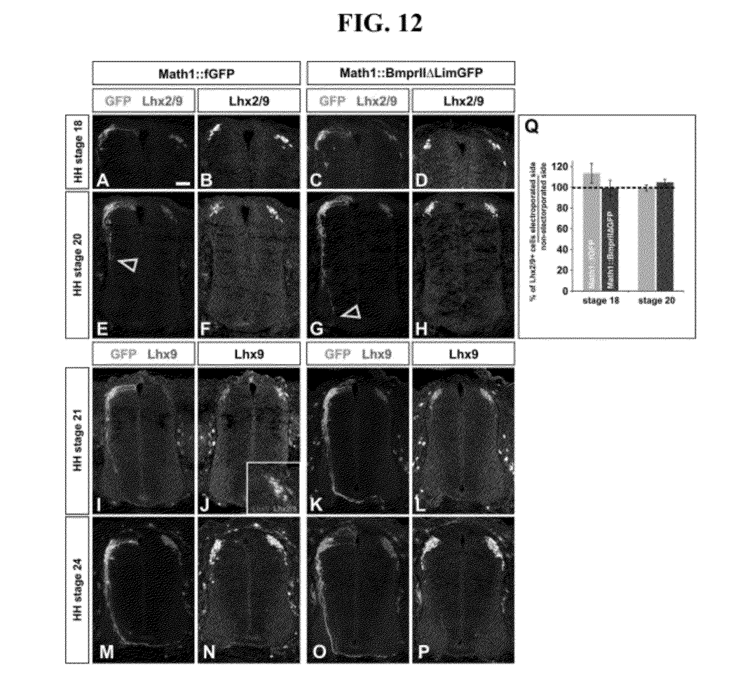

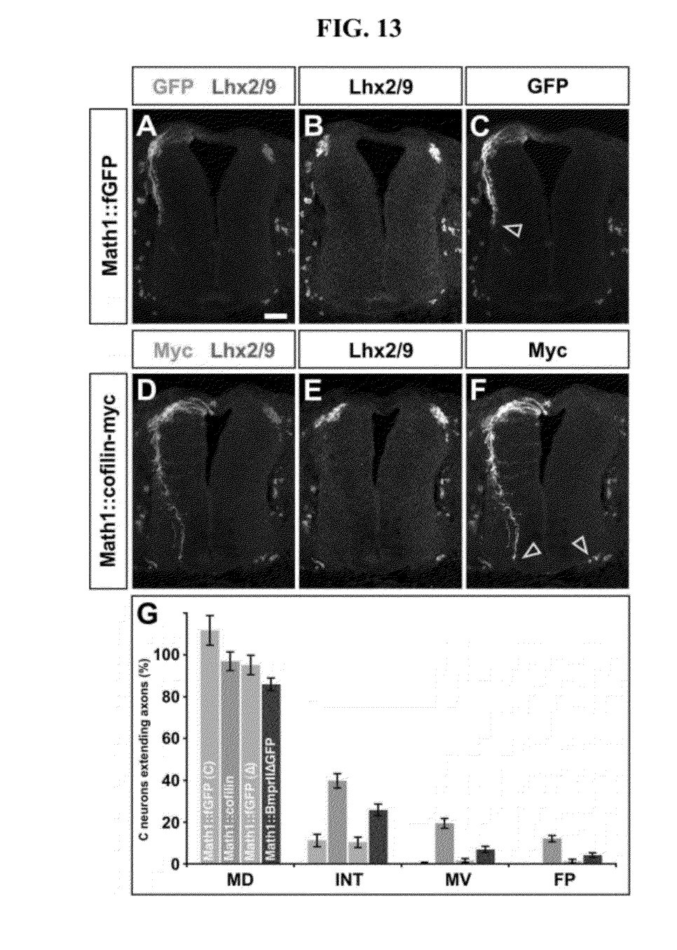

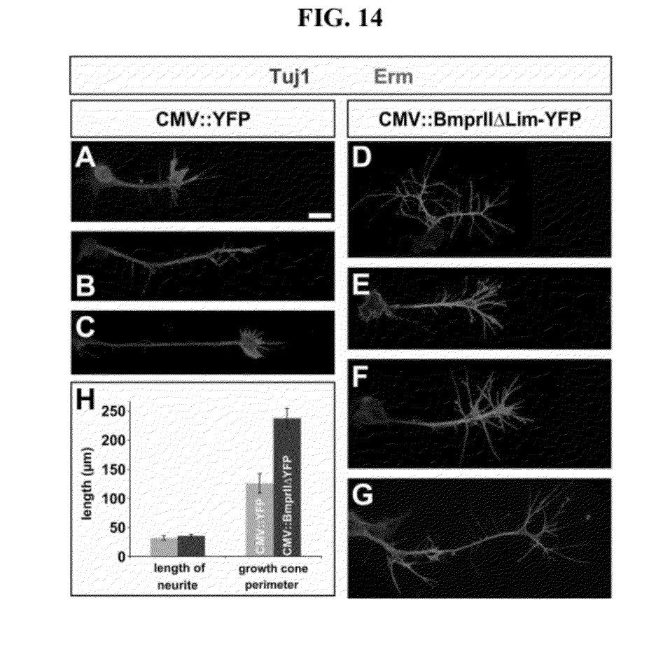

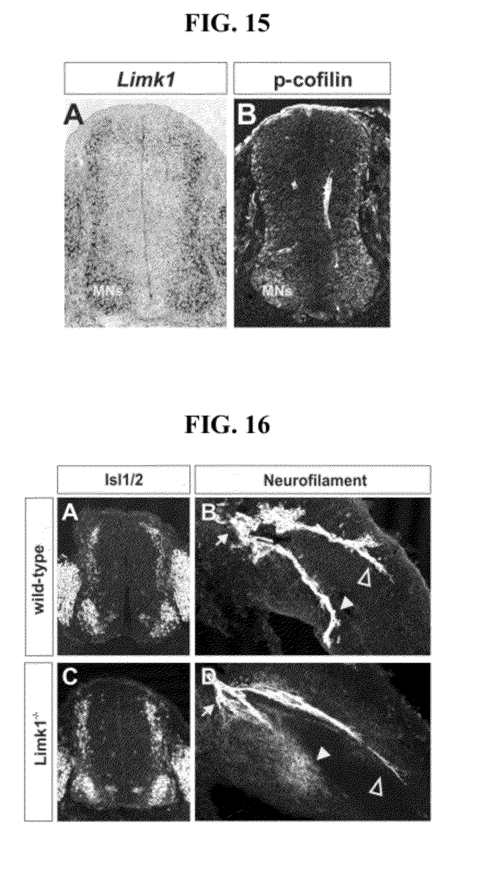

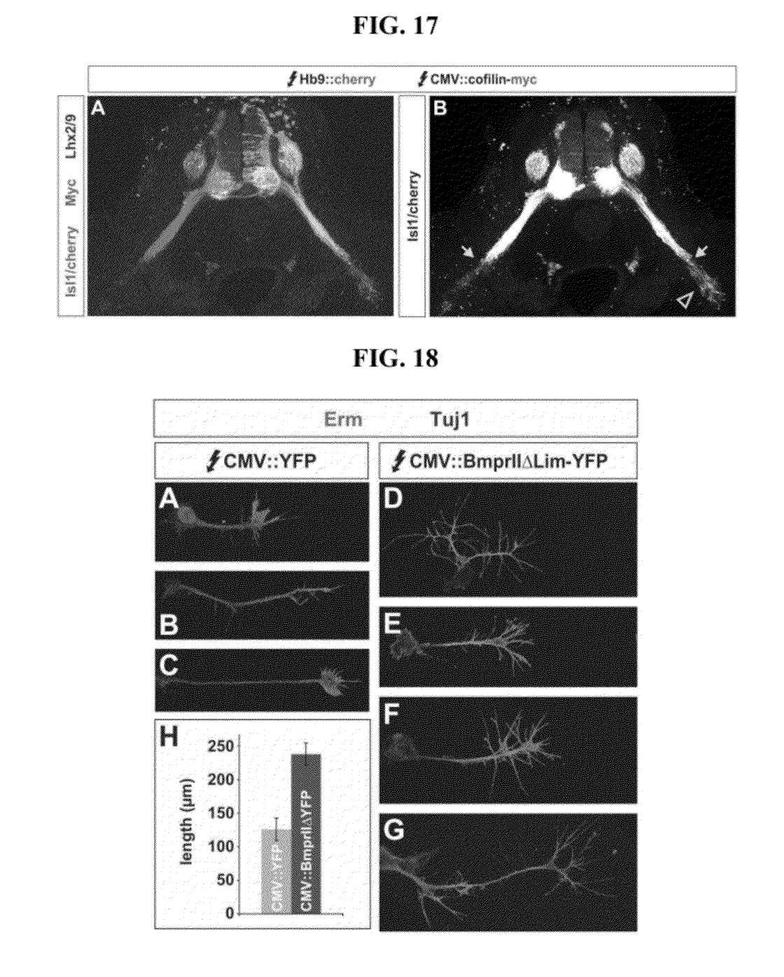

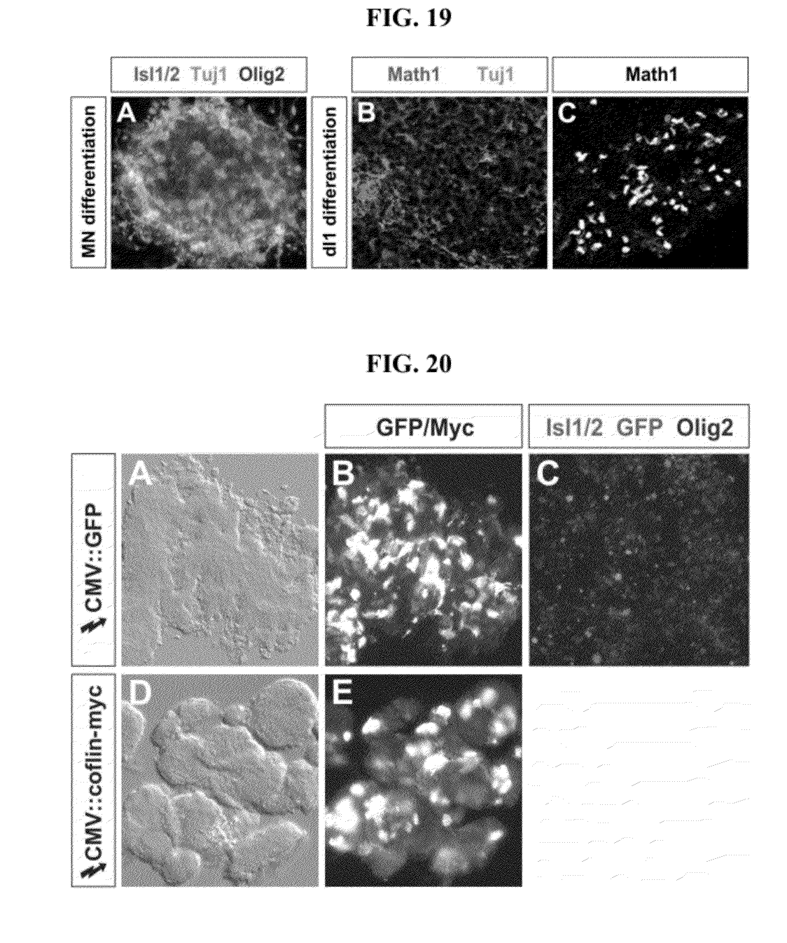

FIG. 1A-B show that Limk1 (A) and phosphorylated (p) cofilin (B) are present in a similar distribution of post mitotic neurons in the E10.5 mouse spinal cord, including the motor neurons (MN).

FIG. 16A-D show aberrant motor axon projections in E11.5 Limk^{-/-} mice. (A, C) Brachial sections of E11.5 wild-type (A) and Limkf′-(C) spinal cords labeled with Isl1/2 antibodies show that the motor columns are at similar stages of development. (B, D) At E11.5 motor axons have segregated at the plexus (arrow, D, B) to innervate the dorsal (open arrowheads, B, D) and ventral (arrowhead, B, D) regions of the limb. The pattern of innervation is abnormal in the Limk^{-/-} limbs (D) compared to controls (B); the dorsal branch appears to be longer, the ventral branch is defasiculated and there are multiple branches emanating from the plexus.

FIG. 17A-B show that overexpressing cofilin in MNs results in accelerated motor axon growth. (A, B) Chicken spinal cords were in ova electroporated first, with Hb9::cherry and CMV::cofilin-myc (experimental) and second. Hb9::cherry (control). By HH stage 24/25 the control and experimental motor axons have extended to the plexus (arrows, B). The control axons have largely paused at the plexus, however, significantly more of the experimental mcy⁺ motor axons have extended past the plexus into the limb (arrowhead, B). Spinal cords were labeled with Isl1/2 (red) and Lhx2/9 (blue) antibodies to show that both sides of the spinal cord were at similar stages of development.

FIG. 18A-G show that Lowering Limk1 activity in MNs results in their extending complex growth cones. (A-G) Dissociated chick MNs electroporated with CMV:: NTH or CMV::Bmprllt1Lim-YF were labeled with antibodies against beta-tubulin (Tuj 1, blue) and the Erm complex (red) to reveal the growth cone morphology. The experimental growth cones (D-G) have longer and more extensive filopodia than the controls (A-C). (H) Quantification of growth cone perimeter.

FIG. 19A-C show neural differentiation of mouse ES cells. (A) EBs containing multiple Olig2⁺ MN progenitor neurons (blue) and mature IsI1/2⁺ MNs (red). Neural processes were labeled with antibodies against beta-tubulin (Tui 1, green). (B, C) EBs containing many Math1⁺ dl1 progenitor neurons (red) and Tag1+ commissural axons (green).

FIG. 20A-E show that electroporation of mouse ES cells (A-E) ES cells were transfected with either CMV::GFP (A-C) or CMV:: cofilin-myc (D, E) and then aggregated into EBs. Control EBs underwent the MN differentiation procedure, however very few GFP′ MNs (i.e. yellow cells) were observed.

### DETAILED DESCRIPTION

It is discovered that the rate of axon extension can be accelerated by overexpressing or activating coli Fin, a direct regulator of the actin cytoekeleon, in neurons. Further, the cofilin-induced extension rate acceleration can be in vivo and local to the extension portion of the neural cell.

The inventors showed that the BMP roof plate (RP) chemorepellent controls the rate of commissural axon outgrowth by activating Limk1. The signal transduction pathway(s) by which morphogens signal to convey guidance information have remained largely unresolved. Here, the inventors link the BMP RP chemorepellent to Limk1/cofilin, intracellular effectors that directly regulate the actin cytoskeleton. Therefore. BMPs from the RP increase Limk1 activity in commissural neurons to control the rate at which they extend axons through their transverse trajectory through the spinal cord.

The ability of the BMP RP chemorepellent to control the rate of axon outgrowth in vivo is a novel activity for the BMPs. It was previously suggested that the BMPs provide a polarizing signal for commissural cell bodies to orient the growth cone away from the dorsal midline. The existence of a polarizing activity remains a possibility, given that loss of BMP signaling disrupts axonal polarity. However, the polarizing activity of the BMPs does not appear to be mediated by Limk1, since the polarity of axon outgrowth remained intact after electroporation with caLimk1-myc (see FIG. 2D, E) and it appears to be either redundant with other signals or weaker than the outgrowth regulating activity of the BMPs in this context.

Without being bound by theory, provided herein is a mechanism by which guidance cues can act to direct axons. Considerable work has focused on understanding how the growth cone interprets directional information; such signals are thought to induce the local rearrangement of the actin cytoskeleton necessary to reorient the growth cone. However, guidance factors, such as the BMPs, may also control the rate at which actin polymerizes in the growth cone to regulate the speed of growth cone extension. The rate of axon growth then determines the response of axons to subsequent guidance cues encountered along their route. In this system, the rate of axon outgrowth is “set” by a preceding guidance cue. Thus, a guidance decision could consist of two components 1) orientation information and 2) rate information to ensure that the growth cone reaches directional signals either at the “right” developmental time or at the “right” speed to correctly interpret subsequent information, in an analogous manner to a car requiring a particular speed to navigate a curve in the road.

In summary, the inventors discovered that the balance of Limk1/cofilin activity acts within a neuron to regulate the speed of axon growth. The present disclosure further shows that by subtly modulating Limk1 activity using BMP signaling, it is possible to “tip” the balance of Limk1/cofilin activity towards promoting axonal growth. Thus, these studies also identify a cell-intrinsic way in which axon outgrowth can be accelerated in vivo. Alteration of this intrinsic pathway can then be used to extend axons in vivo and the extension can be location specific. Similarly, a neural stem cell or neural precursor cell can be extended to provide a valuable source for therapy.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this invention, Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” it also is to be understood, although not always explicitly stated, that the reagents described herein are merely exemplary and that equivalents of such are known in the art.

A “composition” is also intended to encompass a combination of active agent and another carrier, e.g., compound or composition, inert (for example, a detectable agent or label) active, such as an adjuvant, diluent, binder, stabilizer, buffers, salts, lipophilic solvents, preservative, adjuvant or the like. Carriers also include pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

As used herein, the term “patient” intends an animal, a mammal or yet further a human patient. For the purpose of illustration only, a mammal includes but is not limited to a human, a simian, a murine, a bovine, an equine, a porcine or an ovine.

A neuron is an excitable cell in the nervous system that processes and transmits information by electrochemical signaling. Neurons are found in the brain, the vertebrate spinal cord, the invertebrate ventral nerve cord and the peripheral nerves. Neurons can be identified by a number of markers that are listed on-line through the National Institute of Health at the following website: “stemcells.nih.gov/info/scireport/appendixe.asp#eii,” and are commercially available through Chemicon (now a part of Millipore, Temecula, Calif.) or Invitrogen (Carlsbad, Calif.). For example, neurons may be identified by expression of neuronal markers B-tubulin III (neuron marker, Millipore, Chemicon), Tuj1 (beta-III-tubulin); MAP-2 (microtubule associated protein 2, other MAP genes such as MAP-1 or -5 may also be used); anti-axonal growth clones; ChAT (choline acetyltransferase (motoneuron marker, Millipore, Chemicon); Olig2 (motomeuron marker, Millipore, Chemicon), Olig2 (Millipore, Chemicon), CgA (anti-chromagranin A); DARRP (dopamine and cAMP-regulated phosphoprotein); DAT (dopamine transporter); GAD (glutamic acid decarboxylase); GAP (growth associated protein); anti-HuC protein; anti-HuD protein; alpha-internexin; NeuN (neuron-specific nuclear protein); NF (neurofilament); NGF (nerve growth factor); gamma-NSE (neuron specific enolase); peripherin; PH8; PGP (protein gene product); SERT (serotonin transporter); synapsin; Tau (neurofibrillary tangle protein); anti-Thy-1; TRK (tyrosine kinase receptor); TRH (tryptophan hydroxylase); anti-TUC protein; TH (tyrosine hydroxylase); VRL (vanilloid receptor like protein); VGAT (vesicular GABA transporter), VGLUT (vesicular glutamate transporter).

As used herein, “stem cell” defines a cell with the ability to divide for indefinite periods in culture and give rise to specialized cells. Stem cells include, for example, somatic (adult) and embryonic stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell derived from the embryo that has the potential to become a wide variety of specialized cell types. An embryonic stem cell is one that has been cultured under in vitro conditions that allow proliferation without differentiation. Non-limiting examples of embryonic stem cells are the HES2 (also known as ES02) cell line available from ESI, Singapore and the H1 (also know as WA01) cell line available from WiCells, Madison, Wis. In addition, for example, there are 40 embryonic stem cell lines that are recently approved for use in NIH-funded research including CHB-1, CHB-2, CHB-3, CHB-4, CHB-5, CHB-6, CHB-8, CHB-9, CHB-10, CHB-11, CHB-12, RUES1, HUES1, HUES2, HUES3, HUES4, HUES5, HUES6, HUES7, HUES8, HUES9, HUES10, HUES11, HUES12, HUES13, HUES14, HUES15, HUES16, HUES17, HUES18, HUES19. HUES20, HUES21, HUES22, HUES23, HUES24, HUES26, HUES27, and HUES28. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4.

As used herein, a “pluripotent cell” broadly refers to stem cells with similar properties to embryonic stem cells with respect to the ability for self-renewal and pluripotentey (i.e., the ability to differentiate into cells of multiple lineages). Pluripotent cells refer to cells both of embryonic and non-embryonic origin. For example, pluripotent cells includes Induced Pluripotent Stem Cells (iPSCs) or a parthenogenetic stem cell.

An “induced pluripotent stem cell” or “iPSC” or “iPS cell” refers to an artificially derived stem cell from a non-pluripotent cell, typically an adult somatic cell, produced by inducing expression of one or more reprogramming genes or corresponding proteins or RNAs. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Kill. Klf1, Klf2, Klf4 and Klf5; the family of Myc genes, i.e. c-myc and myc; the family of Nanog genes, i.e. OCT4, NANOG and REX1; or LIN28. Examples of iPSCs and methods of preparing them are described in Takahashi et al. Cell 131(5):861-72, 2007; Takahashi & Yamanaka Cell 126:663-76, 2006; Okita et al. Nature 448:260-262, 2007; Yu et al. Science 318(5858):1917-20, 2007; and Nakagawa et al. Nat. Biotechnol. 26(1):101-6, 2008.

A “parthenogenetic stem cell” refers to a stem cell arising from parthenogenetic activation of an egg. Methods of creating a parthenogenetic stem cell are known in the art. See, for example, Cibelli et al. 295(5556):819 (2002) and Vrana et al. 100(Suppl. 1) 11911-6 (2003).

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller Brain Res. 1091(1):258-264, 2006; Pluchino et al. Brain Res. Brain Res. Rev. 48(2):211-219, 2005; and Goh, et al. Stem Cell Res., 12(6):671-679, 2003, Neural stem cells can be identified and isolated by neural stem cell specific markers including, but limited to, CD133, ICAM-1, MCAM, CXCR4 and Notch 1. Neural stem cells can be isolated from animal or human by neural stem cell specific markers with methods known in the art. See, e.g., Yoshida et al., (2006). Stem Cells 24(12):2714-22.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell include, without limitation, a progenitor nerve cell.

A “neural precursor cell”, “neural progenitor cell” or “NP cell” refers to a cell that has a capacity to differentiate into a neural cell or neuron, A NP cell can be an isolated NP cell, or derived from a stem cell including but not limited to an iPS cell. Neural precursor cells can be identified and isolated by neural precursor cell specific markers including, but limited to, nestin and CD133. Neural precursor cells can be isolated from animal or human tissues such as adipose tissue see, e.g., Vindigni et al., (2009) Neurol, Res. 2009 Aug. 5. [Epub ahead of print]) and adult skin (see, e.g., Joannides (2004) Lancet. 364(9429):172-8). Neural precursor cells can also be derived from stem cells or cell lines or neural stem cells or cell lines. See generally, e.g., U.S. Patent Application Publications Nos: 2009/0263901, 2009/0263360 and 2009/0258421.

A nerve cell that is “terminally differentiated” refers to a nerve cell that does not undergo further differentiation in its native state without treatment or external manipulation. In one embodiment, a terminally differentiated cell is a cell that has lost the ability to further differentiate into a specialized cell type or phenotype.

A population of cells intends a collection of more than one cell that is identical (clonal) non-identical in phenotype and/or genotype.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

As used herein, the term “oligonucleotide” or “polynucleotide” refers to a short polymer composed of deoxyribonucleotides, ribonucleotides or any combination thereof. Oligonucleotides are generally at least about 10, 15, 20, 25, 30, 40, 50, 60, 70, 80, 90, 100 or more nucleotides in length. An oligonucleotide may be used as a primer or as a probe.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable where present in its native or natural state, e.g., recombinant replication or manipulation by mutation. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

A “recombinant” nucleic acid refers an artificial nucleic acid that is created by combining two or more sequences that would not normally occur together. In one embodiment, it is created through the introduction of relevant DNA into an existing organism DNA, such as the plasmids of bacteria, to code for or alter different traits for a specific purpose, such as antibiotic resistance. A “recombinant” polypeptide is a polypeptide that is derived from a recombinant nucleic acid.

As used herein, the term “promoter” refers to a nucleic acid sequence sufficient to direct transcription of a gene. Also included in the invention are those promoter elements which are sufficient to render promoter dependent gene expression controllable for cell type specific, tissue specific or inducible by external signals or agents. The term “neuron-specific promoter” refers to a promoter that results in a higher level of transcription of a gene in cells of neuronal lineage compared to the transcription level observed in cells of a non-neuronal lineage. Examples of neuron-specific promoters useful in the methods and compositions described herein include the promoter from neuron-specific enolase (NSE) and the dopamine transporter (DAT).

In some embodiments, a promoter is an inducible promoter or a discrete promoter. Inducible promoters can be turned on by a chemical or a physical condition such as temperature or light. Examples of chemical promoters include, without limitation, alcohol-regulated, tetracycline-regulated, steroid-regulated, metal-regulated and pathogenesis-related promoters. Examples of discrete promoters can be found in, for examples. Wolfe et al. Molecular Endocrinology 16(3): 435-49.

As used herein, the term “regulatory element” refers to a nucleic acid sequence capable of modulating the transcription of a gene. Non-limiting examples of regulatory element include promoter, enhancer, silencer, polyadenylation signal, transcription termination sequence. Regulatory element may be present 5′ or 3′ regions of the native gene, or within an intron.

Various proteins are also disclosed herein with their GenBank Accession Numbers for their human proteins and coding sequences. However, the proteins are not limited to human-derived proteins having the amino acid sequences represented by the disclosed GenBank Accession Nos, but may have an amino acid sequence derived from other animals, particularly, a warm-blooded animal (e.g., rat, guinea pig, mouse, chicken, rabbit, pig, sheep, cow, monkey, etc.).

As used herein, the term “cofilin” or “CFL1” refers to a protein having an amino acid sequence substantially identical to the cofilin sequence of GenBank Accession No. NP_{—}005498. A suitable cDNA encoding cofilin is provided at GenBank Accession. No NM_{—}005507.

As used herein, the term “biological activity of cofilin” refers to any biological activity associated with the full length native cofilin protein. In one embodiment, the biological activity of cofilin refers to binding or depolymerizing filamentous F-actin or inhibiting the polymerization of monomeric G-actin in a pH-dependent manner. In another embodiment, the cofilin biological activity refers to the action of extending axons. In suitable embodiments, the cofilin biological activity is equivalent to the activity of a protein having an amino acid sequence represented by GenBank Accession No. NP_{—}005498. Measurement of transcriptional activity can be performed using any known method, such as immunohistochemistry, reporter assay or RT-PCR.

As used herein, the term “Limk1” or “LIM domain kinase 1” refers to a protein having an amino acid sequence substantially identical to the cofilin sequence of GenBank Accession No. NP_{—}002305. A suitable cDNA encoding cofilin is provided at GenBank Accession No. NM_{—}002314.

As used herein, the term “biological activity of Limk1” refers to any biological activity associated with the full length native Limk1 protein. In one embodiment, the biological activity of Limk1 refers to phosphorylation or inactivation of cofilin. In another embodiment, the Limk1 biological activity refers to the inhibition of axon extension. In suitable embodiments, the Limk1 biological activity is equivalent to the activity of a protein having an amino acid sequence represented by GenBank Accession No. NP_{—}002305. Measurement of transcriptional activity can be performed using any known method, such as immunohistochemistry, reporter assay or RT-PCR.

As used herein, the term “BmprII” or “bone morphogenetic protein receptor, type II” refers to a protein having an amino acid sequence substantially identical to the BmprII sequence of GenBank Accession No. NP_{—}001195. A suitable cDNA encoding BmprII is provided at GenBank Accession No. NM_{—}001204.

As used herein, the term “biological activity of BmprII” refers to any biological activity associated with the full length native BmprII protein. In one embodiment, the biological activity of BmprII refers to phosphorylation or activation of Limk1. In another embodiment, the BmprII biological activity refers to the inhibition of cofilin or axon extension. In suitable embodiments, the BmprII biological activity is equivalent to the activity of a protein having an amino acid sequence represented by GenBank Accession. No. NP_{—}001195. Measurement of transcriptional activity can be performed using any known method, such as immunohistochemistry, reporter assay or RT-PCR.

As used herein, the term “treating” is meant administering a pharmaceutical composition for the purpose of improving the condition of a patient by reducing, alleviating, reversing, or preventing at least one adverse effect or symptom of the disease or condition.

As used herein, the term “preventing” is meant identifying a subject (i.e., a patient) having an increased susceptibility to a disease but not yet exhibiting symptoms of the disease, and administering a therapy according to the principles of this disclosure. The preventive therapy is designed to reduce the likelihood that the susceptible subject will later become symptomatic or that the disease will be delay in onset or progress more slowly than it would in the absence of the preventive therapy. A subject may be identified as having an increased likelihood of developing the disease by any appropriate method including, for example, by identifying a family history of the disease or other degenerative brain disorder, or having one or more diagnostic markers indicative of disease or susceptibility to disease.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, most preferably, a human.

As used herein, the term “substantially identical”, when referring to a protein or polypeptide, is meant one that has at least 80%, 85%, 90%, 95%, or 99% sequence identity to a reference amino acid sequence. The length of comparison is preferably the full length of the polypeptide or protein, but is generally at least 10, 15, 20, 25, 30, 40, 50, 60, 80, or 100 or more contiguous amino acids. A “substantially identical” nucleic acid is one that has at least 80%, 85%, 90%, 95%, or 99% sequence identity to a reference nucleic acid sequence. The length of comparison is preferably the full length of the nucleic acid, but is generally at least 20 nucleotides, 30 nucleotides, 40 nucleotides, 50 nucleotides, 75 nucleotides, 100 nucleotides, 125 nucleotides, or more.

A “biological equivalent” of a polynucleotide or polypeptide sequence refers to a protein or nucleic acid that is substantially identical to the polynucleotide or polypeptide sequence. In one aspect, a biological equivalent of a polynucleotide or polypeptide sequence is one that has shares certain sequence identity with the polynucleotide or polypeptide sequence while still retaining the sequence (e.g., mutation and modification) and/or functional characteristics (e.g., activity and binding specificity) of the polynucleotide or polypeptide sequence. The sequence identity, in one aspect, is at least about 70%, or alternatively at least about 75%, or about 80%, or about 85%, or about 90%, or about 95%, or about 98%, or about 99%.

A “biological equivalent” of SEQ ID NO: 1 (cofilinS3A) includes, without limitation, any mammalian cofilin protein or its biological equivalent, or fragment that is substituted at the Serine at position 3, or an equivalent position, with a non-Serine amino acid such that the protein is not phosphorylated at the serine.

A “biological equivalent” of SEQ ID NO: 2 (BmprIIΔLim) includes, without limitation, any mammalian BmprII protein from which the Lim binding domain is deleted or replaced by another polypeptide. In some embodiments, the deletion or replacement include the 50% sequence at the C-terminus of the wildtype.

As used herein, the term “effective amount” refers to a quantity of compound (e.g., a cofilin protein or biologically active fragment thereof) delivered with sufficient frequency to provide a medical benefit to the patient. In one embodiment, an effective amount of a protein is an amount sufficient to treat or ameliorate a symptom of a neurological disease.

As used here, a “damaged or degenerated neural cell” refers to a neural cell that does not function properly due to a physical damage or degeneration. In one embodiment, the neural cell does not connect to the synaptic target.

A neurological disease characterized by a damaged or a degenerated neural cell, as used herein, refers to a disease having a damaged or a degenerated neural cell. Examples include, without limitation, Traumatic Brain Injury. Alzheimer's disease, Parkinson's disease, epilepsy, Huntington's disease or stroke.

As used herein, “promoting extension of a neural cell” intends increasing the extension of the neural cell at a rate higher than the neural cell would extend at the same location or axon without being treated by a composition or method of the present disclosure. The extension of the neural cell can be measured visually or under microscope by the length or size of the neural cell or a relevant axon. The measurement can alternatively be based on the distance of an axon of the neural cell to the synaptic target.

One embodiment of the present disclosure provides a method for promoting extension of a neural cell, comprising, or alternatively consisting essentially of, or yet alternatively consisting of increasing the biological activity of cofilin in the cell.

In one embodiment, the increasing of the biological activity of cofilin in the cell comprises introducing into the cell an isolated or recombinant cofilin polypeptide or an isolated or recombinant polynucleotide encoding the polypeptide. The isolated or recombinant cofilin, in some aspects, comprises a mutation that inhibits phosphorylation of the cofilin. In another aspect, the isolated or recombinant cofilin comprises the amino acid sequence of SEQ ID NO: (Table 1) or a biological equivalent thereof.

In another embodiment, the increasing of the biological activity of cofilin in the cell comprises inhibiting the expression or the biological activity of Limk1. In the cell. The inhibiting of the activity of Limk1 in the cell, in some aspects, comprises introducing into the cell an isolated or recombinant Limk1 mutant that does not phosphorylate cofilin. In another aspect, the Limk1 mutant does not have one or more of LIM or P1)₇ domain.

In yet another embodiment, the increasing of the biological activity of cofilin in the cell comprises inhibiting the expression or the biological activity of BmprII. The inhibiting of the activity of BmprII, in some aspects, comprises introducing into the cell an isolated or recombinant BmprII mutant that does not phosphorylate Limk1. In another aspect, the BmprII mutant comprises the amino acid sequence of SEQ ID NO: 2 (Table 1) or a biological equivalent thereof.

Other genes are also known to regulate the expression or activity of cofilin, directly or indirectly. For example, nerve growth factor (NGF), netrin1 and F-actin can activate cofilin (Marsick et al., Dev Neurobiol. 70: 565-88, 2010) and so can brain derived growth factor (BDNF) (Chen et al., J Neurobiol. 66: 103-14, 2006). Plexin and semaphoring 7a, in contrast, inactivate cofilin (Scott et al. J Invest Dermatol, 129:954-6, 2009 and Aizawa et al., Nat Neurosci, 4: 367-73, 2001). Other genes that activate the expression or activity of cofilin include, without limitation, Slit2 (Piper et al. Neuron. 49:215-28, 2006), Sonic Hedgehog (shh), slingshot family of phosphotases ssh1, 2 and 3, protein kinase C, Lats1, and RING finger E3 ubiquitin ligase Rrtf6 (Rebecca et al., J. Mol. Med, 85:555-68, 2007). Genes that inactivate or decrease the expression of cofilin include, without limitation, MAPKAPK2, Nogo-66, Nogo-A and Limk2 (Rebecca et al., J. Mol. Med, 85:555-68, 2007 and Hsieh et al., J. Neurosci. 26:1006-15, 2006). The biological activity of cofilin, therefore, can be increased by increasing the biological activity of a gene that activates or increases the expression of cofilin, or alternatively by decreasing the biological activity of a gent that inactivates or decreases the expression of cofilin.

TABLE 1

| SEQ | | |
|---|---|---|
| ID | | |
| NO: | Name | Sequence |
| 1 | cofilinS3A | MAAGVAVSDGVIKVFNDMKVRKSSTPEEVKKRKKAVLFCLSEDKKNIILE |
| | | EGKEILVGDVGQTVDDPYTTFVKMLPDKDCRYALYDATYETKESKKEDLV |
| | | FIFWAPESAPLKSKMIYASSKDAIKKKLTGIKHELQANCYEEVKDRCTLA |
| | | EKLGGSAVISLEGKPL |
| 2 | BmprIIΔLim | MTSSLQRPWRVPWLPWTILLVSTAAASQNQERLCAFKDPYQQDLGIGESR |
| | | ISHENGTILCSKGSTCYGLWEKSKGDINLVKQGCWSHIGDPQECHYEECV |
| | | VTTTPPSIQNGTYRFCCCSTDLCNVNFTENFPPPDTTPLSPPHSFNRDET |
| | | IIIALASVSVLAVLIVALCFGYRMLTGDRKQGLHSMNMMEAAASEPSLDL |
| | | DNLKLLELIGRGRYGAVYKGSLDERPVAVKVFSFANRQNFINEKNIYRVP |
| | | LMEHDNIARFIVGDERVTADGRMEYLLVMEYYPNGSLCKYLSLHTSDWVS |
| | | SCRLAHSVTRGLAYLHTELPRGDHYKPAISHRDLNSRNVLVKNDGTCVIS |
| | | DFGLSMRLTGNRLVRPGEEDNAAISEVGTIRYMAPEVLEGAVNLRDCESA |
| | | LKQVDMYALGLIYWEIFMRCTDLFPGESVPEYQMAFQTEVGNHPTFEDMQ |
| | | VLVSREKQRPKFPEAWKENSLAVRSLKETIEDCWDQDAEARLTAQCAEER |
| | | MAELMMIWERNKSVSPTVNPMSTAMQNER |

In some embodiments, the biological activity of cofilin in the neural cell is increased at a location in the cell proximate to an end of the cell in need of extension, such as an axon.

In some embodiments, the neural cell is a neural stem cell. The neural stem cell can be derived from an induced pluripotent stem cell (iPSC), an embryonic stem cell or a parthenogenetic stem cell. In another embodiment, the neural cell is a neural precursor cell.

In some embodiments, the neural cell is a damaged or degenerated neural cell that is terminally differentiated.

Yet still in some embodiments, the increasing of the biological activity of cofilin is in vivo or ex vivo. In one embodiment, the neural cell is a mammalian neural cell. For the purpose of illustration only, a mammal includes but is not limited to a simian, a murine, a bovine, an equine, a porcine, an avian or an ovine.

Methods of increasing the biological activity of a gene or protein are known in the art and are further described below.

Methods for Increasing the Level of a Protein in a Cell

Methods for increasing the level of a protein, or polypeptide or peptide, in a cell are known in the art. In one aspect, the protein level is increased by increasing the amount of a polynucleotide encoding the protein, wherein that polynucleotide is expressed such that new protein is produced. In another aspect, increasing the protein level is increased by increasing the transcription of a polynucleotide encoding the protein, or alternatively translation of the protein, or alternatively post-translational modification, activation or appropriate folding of the protein. In yet another aspect, increasing the protein level is increased by increasing the binding of the protein to appropriate cofactor, receptor, activator, ligand, or any molecule that is involved in the protein's biological functioning. In some embodiments, increasing the binding of the protein to the appropriate molecule is increasing the amount of the molecule. In one aspect of the embodiments, the molecule is a protein. In another aspect of the embodiments, the molecule is a small molecule. In a further aspect of the embodiments, the molecule is a polynucleotide.

Methods of increasing the amount of polynucleotide encoding the protein in a cell are known in the art. In one aspect, the polynucleotide can be introduced to the cell and expressed by a gene delivery vehicle that can include a suitable expression vector.

Suitable expression vectors are well-known in the art, and include vectors capable of expressing a polynucleotide operatively linked to a regulatory element, such as a promoter region and/or an enhancer that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

Non-viral vector may include plasmid that comprises a heterologous polynucleotide capable of being delivered to a target, cell, either in vitro, in vivo or ex-vivo. The heterologous polynucleotide can comprise a sequence of interest and can be operably linked to one or more regulatory elements and may control the transcription of the nucleic acid sequence of interest. As used herein, a vector need not be capable of replication in the ultimate target cell or subject. The term vector may include expression vector and cloning vector.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. In aspects where gene transfer is mediated by a retroviral vector, a vector construct refers to the polynucleotide comprising the retroviral genome or part thereof, and a therapeutic gene. As used herein, “retroviral mediated gene: transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant. Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells or cardiomyoeytes. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

Proteins have been described that have the ability to translocate desired nucleic acids across a cell membrane of the nerve cell. Typically, such proteins have amphiphilic or hydrophobic subsequences that have the ability to act as membrane-translocating carriers. For example, homeodomain proteins have the ability to translocate across cell membranes. The shortest internalizable peptide of a homeodomain protein, Antennapedia, was found to be the third helix of the protein, from amino acid position 43 to 58 (see, e.g., Prochiaritz, 1996, Current Opinion in Neurobiology 6:629-634. Another subsequence, the h (hydrophobic) domain of signal peptides, was found to have similar cell membrane translocation characteristics (see, e.g., Lin et al., 1995, J. Biol. Chem. 270:14255-14258). Such subsequences can be used to translocate oligonucleotides across a cell membrane. Oligonucleotides can be conveniently derivatized with such sequences. For example, a linker can be used to link the oligonucleotides and the translocation sequence. Any suitable linker can be used, e.g., a peptide linker or any other suitable chemical linker.

Methods of delivering a protein to a cell, either to increase the biological activity of itself or a protein positively regulated by this protein, or to decrease the biological activity of a protein negatively regulated by this protein, are generally known in the art. For example, proteins can be delivered to a eukarotic cell by a type III sercreation machine. See, e.g., Galan and Wolf-Watz. (2006) Nature 444:567-73. Biologically active and full length protein, for another example, can also be delivered into a cell using cell penetraint peptides (CPP) as delivery vehicles. The trans-activating transcriptional activator (TAT) from human immunodeficiency virus 1 (HIV-1) is such a CPP, which is able to deliver different proteins, such as horseradish peroxidase and RNase A across cell membrane into the cytoplasm in different cell lines. Wadia et al. (2004) Nat. Med 10:310-15. Accordingly, in one aspect, a protein, such as cofilin, can be delivered to a neural precursor cell using TAT as a vehicle to increase the biological activity of cofilin in the cell.

Liposomes, microparticles and nanoparticles are also known to be able to facilitate delivery of proteins or peptides to a cell (reviewed in Tan et al., (2009) Peptides 2009 Oct. 9. [Epub ahead of print]). The liposomes, microparticles or nanoparticles can also comprise a targeting antibody or fragment thereof can be used in the methods of this invention. To enhance delivery to a cell, the proteins can be conjugated to antibodies or binding fragments thereof which bind cell surface antigens, e.g., a cell surface marker found on progentior cells.

In another aspect, non-covalent method which forms CPP/protein complexes has also been developed to address the limitations in covalent method such as chemical modification before crosslinking and denaturation of proteins before delivery. For example, a short amphipathic peptide carrier, Pep-1 and protein complexes have proven effective for delivery. It was shown that Pep-1 could facilitate rapid cellular uptake of various peptides, proteins and even full-length antibodies with high efficiency and less toxicity. Cheng et al. (2001) Nat. Biotechnol. 19:1173-6.

Proteins can be synthesized for delivery. Nucleic acids that encode a protein or fragment thereof may be introduced into various cell types or cell-free systems for expression, thereby allowing purification of cofilin or other proteins, for large-scale production and patient therapy.

Eukaryotic and prokaryotic expression systems may be generated in which a gene sequence is introduced into a plasmid or, other vector, which is then used to transform living cells. Constructs in which the cDNA contains the entire open reading frame inserted in the correct orientation into an expression plasmid may be used for protein expression. Prokaryotic and eukaryotic expression systems allow for the protein to be recovered, if desired, as fusion proteins or further containing a label useful for detection and/or purification of the protein. Typical expression vectors contain regulatory elements that direct the synthesis of large amounts mRNA corresponding to the inserted nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria, such as Escherichia coli, requires the insertion of the nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Stable or transient cell line clones of mammalian cells can also be used to express a protein. Appropriate cell lines include, for example, COS, HEK293T, CHO, or NIH cell lines.

Once the appropriate expression vectors containing a gene, fragment, fusion, or mutant are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals (e.g., mammals). In vitro expression of a protein, fusion, polypeptide fragment, or mutant encoded by cloned DNA may also be used. Those skilled in the art of molecular biology will understand that a wide variety of expression systems and purification systems may be used to produce recombinant proteins and fragments thereof.

Once a recombinant protein is expressed, it can be isolated from cell lysates using protein purification techniques such as affinity chromatography. Once isolated, the recombinant protein can, if desired, be purified further by e.g., by high performance liquid chromatography (HPLC; e.g., see Fisher, Laboratory Techniques In Biochemistry And Molecular Biology, Work and Burdon, Eds., Elsevier, 1980).

### Vectors Suitable for Delivery to Humans

This disclosure features methods and compositions for extending axons. In one aspect, the invention features methods of gene therapy to express a gene or protein in a neural cell of a patient. Gene therapy, including the use of viral vectors as described herein, seeks to transfer new genetic material (e.g., polynucleotides encoding cofilin or other proteins or a biologically active fragment thereof) to the cells of a patient with resulting therapeutic benefit to the patient.

For in vivo gene therapy, expression vectors encoding the gene of interest is administered directly to the patient. The vectors are taken up by the target cells (e.g., neurons or pluripotent stem cells) and the gene expressed. Recent reviews discussing methods and compositions for use in gene therapy include Eck et al., in Goodman & Gilman's The Pharmacological Basis of Therapeutics, Ninth Edition, Hardman et al., eds., McGray-Hill, N.Y., 1996, Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, and the references cited therein. U.S. Pat. No. 6,080,728 also provides a discussion of a wide variety of gene delivery methods and compositions.

Adenoviruses are able to transfect a wide variety of cell types, including non-dividing cells. There are more than 50 serotypes of adenoviruses that are known in the art, but the most commonly used serotypes for gene therapy are type 2 and type 5. Typically, these viruses are replication-defective; and genetically-modified to prevent unintended spread of the virus. This is normally achieved through the deletion of the E1 region, deletion of the E1 region along with deletion of either the E2 or E4 region, or deletion of the entire adenovirus genome except the cis acting inverted terminal repeats and a packaging signal (Gardlik et al., Med Sci Monit. 11: RA110-121, 2005).

Retroviruses are also useful as gene therapy vectors and usually (with the exception of lentiviruses) are not capable of transfecting non-dividing cells. Accordingly, any appropriate type of retrovirus that is known in the art may be used, including, but not limited to, HIV, SIV, FTV, DAV, and Moloney Murine Leukaemia Virus (MoMLV). Typically, therapeutically useful retroviruses including deletions of the gag, poi, or env genes.

In another aspect, the invention features the methods of gene therapy that utilize a lentivirus vectors to express cofilin, or other proteins in a patient. Lentiviruses are a type of retroviruses with the ability to infect both proliferating and quiescent cells. An exemplary lentivirus vector for use in gene therapy is the HIV-1 lentivirus. Previously constructed genetic modifications of lentiviruses include the deletion of all protein encoding genes except those of the gag, poi, and rev genes (Moreau-Gaudry et al. (2001) Blood. 98: 2664-2672).

Adeno-associated virus (AAV) vectors can achieve latent infection of a broad range of cell types, exhibiting the desired characteristic of persistent expression of a therapeutic gene in a patient. The invention includes the use of any appropriate type of adeno-associated virus known in the art including, but not limited to AAV1, AAV2, AAV3, AAV4, AAV5, and AAV6 (Lee et al., (2205) Biochem J. 387: 1-15; U.S. Patent Publication 2006/0204519).

Herpes simplex virus (HSV) replicates in epithelial cells, but is able to stay in a latent state in non-dividing cells such as the midbrain dopaminergic neurons. The gene of interest may be inserted into the LAT region of HSV, which is expressed during latency. Other viruses that have been shown to be useful in gene therapy include parainfluenza viruses, poxviruses, and alphaviruses, including Semliki forest virus, Sinbis virus, and Venezuelan equine encephalitis virus (Kennedy (1997) Brain. 120:1245-1259).

Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In vivo DNA-mediated gene transfer into a variety of different target sites has been studied extensively. Naked DNA may be administered using an injection, a gene gun, or electroporation. Naked DNA can provide long-term expression in muscle. See Wolff, et al., (1992) Human Mol. Genet, 1:363-369; Wolff, et al. (1990) Science 247:1465-1468. DNA-mediated gene transfer has also been characterized in liver, heart, lung, brain and endothelial cells. See Zhu, et al. (1993) Science 261:209-211; Nabel et al. (1989) Science 244:1342-1344. DNA for gene transfer also may be used in association with various cationic lipids, polycations and other conjugating substances. See Przybylska et al. (2004) J. Gene Med. 6:85-92; Svahn et al. (2004) J. Gene Med. 6:S36-S44.

Methods of gene therapy using cationic liposomes are also well known in the art. Exemplary cationic liposomes for use in this invention are DOTMA, DOPE, DOSPA, DOTAP, DC-Chol, Lipid GL-67™, and EDMPC. These liposomes may be used in vivo or ex vivo to encapsulate a vector for delivery into target cells (e.g., neurons or pluripotent stem cells).

Typically, vectors made in accordance with the principles of this disclosure will contain regulatory elements that will cause constitutive expression of the coding sequence. Desirably, neuron-specific regulatory elements such as neuron-specific promoters are used in order to limit or eliminate ectopic gene expression in the event that the vector is incorporated into cells outside of the target region. Several regulatory elements are well known in the art to direct neuronal specific gene expression including, for example, the neural-specific enolase (NSE), and synapsin-1 promoters (Morelli et al. (1999) J. Gen. Virol. 80:571-583).

### Direct Protein Administration

The level of a protein also may be increased in cells by directly administering that protein to the cells in a manner in which the protein is taken up by the cell (i.e., transits across the cell membrane into the cytoplasm). To help facilitate the delivery of any protein into a cell and across the cell membrane, the protein may be fused chemically or recombinantly, or otherwise associated with a peptide that facilitates the delivery, such as a cell penetrating peptides (CPP) or protein transduction domain (PTD).

Cell penetrating peptides, or “CPPs”, as used herein, refer to short peptides that facilitate cellular uptake of various molecular cargos (from small chemical molecules to nanosize particles and large fragments of DNA), A “cargo”, such as a protein, is associated with the peptides either through chemical linkage via covalent bonds or through non-covalent interactions. The function of the CPPs are to deliver the cargo into cells, a process that commonly occurs through endocytosis with the cargo delivered to the endosomes of living mammalian cells. CPPs typically have an amino acid composition containing either a high relative abundance of positively charged amino acids such as lysine arginine, or have sequences that contain an alternating pattern of polar/charged amino acids and non-polar, hydrophobic amino acids. In 1988, Frankel and Pubo found that the human immunodeficiency virus transactivator of transcription (HIV-TAT) protein can be delivered to cells using a CPP (Frankel et al., (1988) Cell 55(6):1189-1193 and Frankel et al. (1988) Science 240:70-73).

A CPP employed in accordance with one aspect of the invention may include 3 to 35 amino acids, preferably 5 to 25 amino acids, more preferably 10 to 25 amino acids, or even more preferably 15 to 25 amino acids.

A CPP may also be chemically modified, such as prenylated near the C-terminus of the CPP. Prenylation is a post-translation modification resulting in the addition of a 15 (farneysyl) or 20 (geranylgeranyl) carbon isoprenoid chain on the peptide. A chemically modified CPP can be even shorter and still possess the cell penetrating property. Accordingly, a CPP, pursuant to another aspect of the invention, is a chemically modified CPP with 2 to 35 amino acids, preferably 5 to 25 amino acids, more preferably 10 to 25 amino acids, or even more preferably 15 to 25 amino acids.

A CPP suitable for carrying out one aspect of the invention may include at least one basic amino acid such as arginine, lysine and histidine. In another aspect, the CPP may include more, such as 2, 3, 4, 5, 6, 7, 8, 9, 10, or more such basic amino acids, or alternatively about 5%, 10%, 15%, 20%, 25%, 30%, 40%, 50% of the amino acids are basic amino acids. In one embodiment, the CPP contains at least two consecutive basic amino acids, or alternatively at least three, or at least five consecutive basic amino acids. In a particular aspect, the CPP includes at least two, three, four, or five consecutive arginine. In a further aspect, the CPP includes more arginine than lysine or histidine, preferably includes more arginine than lysine and histidine combined.

CPPs may include acidic amino acids but the number of acidic amino acids should be smaller than the number of basic amino acids. In one embodiment, the CPP includes at most one acidic amino acid. In a preferred embodiment, the CPP does not include acidic amino acid. In a particular embodiment, a suitable CPP is the HIV-TAT peptide.

CPPs can be linked to a protein recombinantly, covalently or non-covalently. A recombinant protein having CPP peptide can be prepared in bacteria, such as E. coli, a mammalian cell such as a human HEK293 cell, or any cell suitable for protein expression. Covalent and non-covalent methods have also been developed to form CPP/protein complexes, A CPP, Pep-1, has been shown to form a protein complex and proven effective for delivery (Kameyama et al. (2006) Bioconjugate Chem. 17:597-602).

CPPs also include cationic conjugates which also may be used to facilitate delivery of the proteins into the progenitor or stem cell. Cationic conjugates may include a plurality of residues including amines, guanidines, anticlines, N-containing heterocycles, or combinations thereof. In related embodiments, the cationic conjugate may comprise a plurality of reactive units selected from the group consisting of alpha-amino acids, beta-amino acids, gamma-amino acids, canonically functionalized monosaccharides, canonically functionalized ethylene glycols, ethylene imines, substituted ethylene imines, N-substituted spermine, N-substituted spermidine, and combinations thereof. The cationic conjugate also may be an oligomer including an oligopeptide, oligoamide, canonically functionalized oligoether, canonically functionalized oligosaccharide, oligoamine, oligoethyleneimine, and the like, as well as combinations thereof. The oligomers may be oligopeptides where amino acid residues of the oligopeptide are capable of forming positive charges. The oligopeptides may contain 5 to 25 amino acids; preferably 5 to 15 amino acids; more preferably 5 to |0 cationic amino acids or other cationic subunits.

Recombinant proteins anchoring CPP to the proteins can be generated to be used for delivery to neural progenitor cells or stem cells to prepare mature and functional DA neurons.

Accordingly, in one aspect, the invention provides a method for promoting the extension of a neural cell or neural stem cells by contacting the cell with at least one protein of cofilin, a non-phosphorylatable mutant of coffin, Limk1 mutant protein that does not phosphorylate cofilin, or a BmprII mutant protein that does not phosphorylate Preferably, each of the proteins is attached to a CPP.

### Pharmaceutical or Therapeutic Compositions

The invention, in another aspect, provides a neural cell or cell population produced by the methods of the invention as disclosed herein. In one aspect, the cell population is a purified or isolated substantially homogeneous population of cells.

The present disclosure further provides, in one embodiment, a neural cell comprising, or alternatively consisting essentially of, or yet further consisting of an isolated or recombinant polypeptide comprising an amino acid sequence of SEQ ID NO: 1, 2 or a biological equivalent thereof, or an isolated or recombinant polynucleotide comprising, or alternatively consisting essentially of, or yet further consisting of a nucleic acid sequence encoding SEQ ID NO: 1, 2 or a biological equivalent thereof.

In one embodiment, the polypeptide or polynucleotide is localized location in the cell proximate to an end of the cell in need of extension.

In another embodiment, the neural cell is a neural stem cell or a neural precursor cell. The neural stem cell can be derived from an induced pluripotent stem cell (iPSC), an embryonic stem cell or a parthenogenetic stem cell.

In another embodiment, the neural cell is a damaged or degenerated neural cell that is terminally differentiated. Further provided, in one embodiment, is an isolated population of any of the above neural cells. In one aspect, the cell population is a purified or isolated substantially homogeneous population of cells.

In yet another aspect, the invention provides a pharmaceutical composition comprising a neural cell produced by the methods of the invention and a pharmaceutically acceptable carrier or excipient.

The present invention also includes the administration of therapeutic molecules, such as polynucleotides, proteins or small molecules to a subject. The therapeutic molecules can be administered to a subject, e.g., a human, alone or in combination with any pharmaceutically acceptable carrier or salt known in the art. Pharmaceutically acceptable salts may include non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acids such as hydrochloric acid, hydrobromic acid, sulfuric acid phosphoric acid, or the like. Metal complexes include zinc, iron, and the like. Exemplary pharmaceutically acceptable carriers include physiological saline and artificial cerebrospinal fluid (aCSF). Other physiologically acceptable carriers and their formulations are known to one skilled in the art and described, fOr example, in Remington: The Science and Practice of Pharmacy, (21st edition), 2005, Lippincott Williams & Wilkins Publishing.

Pharmaceutical formulations of a therapeutically effective amount of a compound of the invention, or pharmaceutically acceptable salt-thereof, can be administered parenterally (e.g. intramuscular, intraperitoneal, intravenous, or subcutaneous injection), or by intrathecal intracerebroventricular injection in an admixture with a pharmaceutically acceptable carrier adapted for the route of administration.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the proteins of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

The protein or therapeutic compound can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or subacute disorder, a treatment with an immediate release form will be preferred over a prolonged release composition. Alternatively, for preventative or long-term treatments, a sustained released composition will generally be preferred.

### Treatments

In one embodiment, the present disclosure provides a method for treating a neurological disease characterized by a damaged or a degenerated neural cell, comprising increasing the biological activity of cofilin in the neural cell to promote the extension of the neural cell, thereby treating the disease.

In one embodiment, the present disclosure provides a method for treating, a neurological disease characterized by a damaged or a degenerated neural cell, comprising introducing to the neural cell an isolated or recombinant polypeptide comprising, or alternatively consisting essentially of, or yet further consisting of, an amino acid sequence of SEQ ID NO: 1, 2 or a biological equivalent thereof, or an isolated or recombinant polynucleotide comprising, or alternatively consisting essentially of, or yet further consisting of, a nucleic acid sequence encoding SEQ ID NO: 1, 2 or a biological equivalent thereof.

In one embodiment, the present disclosure provides a method for treating a neurological disease characterized by a damaged or a degenerated neural cell in a subject, comprising or alternatively consisting essentially of, or yet consisting, implanting into the subject any of the above neural cells or isolated or purified population of cells.

A neurological characterized by a damaged or a degenerated neural cell includes, for example without limitation, Traumatic Brain Injury, Alzheimer's disease, Parkinson's disease, epilepsy, Huntington's disease or stroke.

### Transplantation of Extended Neural or Neural Stem Cells

In another aspect, ex vivo gene therapy is used to effect gene expression in a neuron of a patient. Generally, this therapeutic strategy involves using the expression vectors and techniques described above to transfect cultured cells in vitro prior to implantation of those cells into the neuron of a patient. The advantage of this strategy is that the clinician can ensure that the cultured cells are expressing suitable levels of genes in a stable and predictable manner prior to implantation. Such preliminary characterization also allows for more precise control over the final dosage of proteins that will be expressed by the modified cells.

In one embodiment, autologous cells are isolated, transfected, and implanted into the patient. The use of autologous cells minimizes the likelihood of rejection or other deleterious immunological host reaction. Other useful cell types include, for example, pluripotent stem cells, including umbilical cord blood stem cells, neuronal progenitor cells, fetal mesencephalic cells, embryonic stem cells, and postpartum derived cells (U.S. Patent Application 2006/0233766). In another embodiment, cells are encapsulated in a semipermeable, microporous membrane and transplanted into the patient adjacent to the substantia nigra (WO 97/44065 and U.S. Pat. Nos. 6,027,721; 5,653,975; 5,639,275), the caudate, and/or the putamen. The encapsulated cells are modified to express a secreted version of encoded proteins in order to provide a therapeutic benefit to the surrounding brain regions. The secreted proteins may be native proteins, biologically active protein fragments, and/or modified proteins which have increased cell permeability relative to the native proteins (e.g., proteins fused to a CPP).

Cell transplantation therapies typically involve grafting the replacement cell populations into the lesioned region of the nervous system (e.g., the A9 region of the substantia nigra, the caudate, and/or the putamen), or at a site adjacent to the site of injury. Most commonly, the therapeutic cells are delivered to a specific site by stereotaxic injection. Conventional techniques for grafting are described, for example, in Bjorklund et al. (Neural Grafting in the Mammalian CNS, eds. Elsevier, pp 169-178, 1985), Leksell et al. (Acta Neurochir. (1980) 52:1-7) and Leksell et al. (J. Neurosurg. (1987) 66:626-629). Identification and localization of the injection target regions will generally be done using a non-invasive brain imaging technique (e.g., MRI) prior to implantation (see, for example, Leksell et al. (1985) J. Neurol, Neurosurg, Psychiatry 48:14-18).

Briefly, administration of cells into selected regions of a patient may be made by drilling a hole and piercing the dura to permit the needle of a microsyringe to be inserted. The cell preparation permits grafting of the cells to any predetermined site in the brain or spinal cord. It also is possible to effect multiple grafting concurrently, at several sites, using the same cell suspension, as well as mixtures of cells. Multiple graftings may be unilateral, bilateral, or both. Typically, grafting into larger brain structures such as the caudate and/or putamen will require multiple graftings at spatially distinct locations.

Following in vitro cell culture and isolation as described herein, the cells are prepared for implantation. The cells are suspended in a physiologically compatible carrier, such as cell culture medium (e.g., Eagle's minimal essential media), phosphate buffered saline, or artificial cerebrospinal fluid (aCSF). Cell density is generally about 10⁷ to about 10⁸ cells/ml. The volume of cell suspension to be implanted will vary depending on the site of implantation, treatment goal, and cell density in the solution. For the treatment of Parkinson's Disease, for example, about 30-100 μl of cell suspension will be administered in each intra-nigral or intra-putamenal injection and each patient may receive a single or multiple injections into each of the left and right nigral or putaminal regions.

In some embodiments, the cells expressing cofilin or other proteins are encapsulated within permeable membranes prior to implantation. Encapsulation provides a barrier to the host's immune system and inhibits graft rejection and inflammation. Several methods of cell encapsulation may be employed. In some instances, cells will be individually encapsulated. In other instances, many cells will be encapsulated within the same membrane. Several methods of cell encapsulation are well known in the art, such as described in European Patent Publication No. 301,777, or U.S. Pat. Nos. 4,353,888, 4,744,933, 4,749,620, 4,814,274, 5,084,350, and 5,089,272.

In one method of cell encapsulation, the isolated cells are mixed with sodium alginate and extruded into calcium chloride so as to form gel beads or droplets. The gel beads are incubated with a high molecular weight (e.g., MW 60-500 kDa) concentration (0.03-0.1% w/v) polyamino acid (e.g., poly-L-lysine) to form a membrane. The interior of the formed capsule is re-liquefied using sodium citrate. This creates a single membrane around the cells that is highly permeable to relatively large molecules (MW ˜200-400 kDa), but retains the cells inside. The capsules are incubated in physiologically compatible carrier for several hours in order that the entrapped sodium alginate diffuses out and the capsules expand to an equilibrium state. The resulting alginate-depleted capsules is reacted with a low molecular weight polyamino acid which reduces the membrane permeability (MW cut-off ˜40-80 kDa).

### Identification of Candidate Compounds Useful for Extending Neural Cells

A candidate compound that is beneficial for promoting extension of axons of a neural or neural stem cell can be identified using the methods described herein. A candidate compound can be identified for its ability to increase the expression or biological activity of cofilin or decrease the expression or biological activity of Limk1 or BmprII. Candidate compounds that modulate the expression level or biological activity of the protein by at least 10%, 20%, 30%, 40%, 50%, 60%, 70%, 80%, 90%, 95%, 100%, or more relative to an untreated control not contacted with the candidate compound are identified as compounds useful for promoting extension of neural cells and useful for treating neurological diseased characterized by damaged or degenerated neurons.

### Kits

Also provided are kits for use in promoting extension of a neural cell, comprising an isolated or recombinant polypeptide comprising an amino acid sequence of SEQ ID NO: 1, 2 or a biological equivalent thereof, or an isolated or recombinant polynucleotide comprising a nucleic acid sequence encoding, SEQ ID NO: 1, 2 or a biological equivalent thereof and instructions to use.

Still further provided, in one embodiment, is a kit for use in treating a neurological disease characterized by a damaged or a degenerated neural cell, comprising, or alternatively consisting essentially of, or yet further consisting of any neural cell or population of cells, of the above embodiments and instructions to use.

### EXAMPLES

### Example 1

This example demonstrates that inhibition of cofilin by constitutively activation of Limk1 leads to stalled commissural axon outgrowth whereas lowered Limk1 activity accelerates axon outgrowth.

### Materials and Methods

### Immununohistochemistry and In Situ Hybridization

Antibody staining and in situ hybridization histochemistry was performed on either cryosectioned or whole mount tissues as previously described (Augsburger et al., 1999). Fluorescence and'DIC images were collected on a Zeiss LSM510 confocal and Axiovert 200M and Axioplan 2 microscopes. Images were processed using Adobe Photoshop CS2 and CS4.

The antibodies against the following proteins were used: mouse: phosphorylated-cofilin at 1:500 (Cell Signaling Technology), neuronal class III β-Tubulin at 1:1000 (Tuj1, Covance Inc.), GFP at 1:2000 (3E6, Invitrogen), His at 1:1000 (Covance), Erm at 1: 100 (13H9, (Birgbauer and Solomon, 1989) Myc at 1:1000 (9E10 (Evan et al., 1985)); rabbit: cofilin at 1:500 (Cytoskeleton panLh2 (Lhx2/9) at 1:2000 (L1, (Liern et al., 1997), panIs1 (Isl1/2) at 1:2000 (K5, (tient et al. 1997), axonin1 at 1:10,000 (Ruegg et al., 1989); guinea pig: Lhx2 at 1:2000, (Lee et al., 1998), Lhx9 at 1:500 (Lee et al., 1998). Cy3-, Cy5- or FITC-coupled secondary antibodies were obtained from Jackson Ininiunoresearch.

An in situ probe against the 3′ UTR of the mouse Limk1 mRNA was prepared using the following primers: forward, 5′-AGGGATCTGAATCCCCAAAC-3′ (SEQ ID NO: 3), reverse 5′-GAGATTAACCCTCACTAAAGGAACAATCCCATCCCCCFAAA C-3 (SEQ ID NO: 4). The underlined region denotes a T3 polymerase site embedded in the primer sequence. The target sequence was amplified from E10.5 mouse spinal cord cDNA by PCR. Qiaquick (Qiagen) purified products were used in an in vitro transcription reaction using the Roche DIG RNA labeling kit.

### Generation and Analysis of Expression Constructs

Math1 enhancer expression constructs were generated as previously described (Yamauchi et al., 2008) by replacing the lacZ reporter gene in the BGZA vector with hill-length rat cofilin, a non-phosphorylatable form of rat cofilin, cofilinS3A, (Arber et al., 1998) and a truncated form (k1) of mouse Limk1 that is constitutively active (Arber et al., 1998). The BmprIIΔLim-GFP and BmprIIΔLim-YFP fusion proteins were generated by replacing amino acids 530 to 1039 encompassing the Lim binding domain in the carboxy-terminus of human BmprII (Rosenzweig et al., 1995) with eGFP or Venus-YR respectively as follows: using upstream (5′-GCCGCCACATGTCITCCTCGCTGCAGCGGCC-3′ SEQ ID NO: 5) and downstream (5′-GCCGCCACTAGTGACAGGTTGCGTTCATTCTGCA-3′ SEQ ID NO: 6) primers, 1-1587 base pairs of the extracellular domain of BmprII were amplified by PCR using the full length receptor as a template. This amino-terminal fragment of BmprII was fused to eGFP and inserted into the BGZA vector as above. In ovo electroporation was performed as previously described (Yamauchi et al., 2008). Stage HH stage 15 chicken embryos (AA Laboratory Eggs) were electroporated with a range of concentrations of the Math1 expression plasmids: 0.6-1 μg/μl BmprIIΔLim-GFP, 0.2 μg/μl fGFP, 1.0 μg/μl cofilin-myc, 1.0 μg/μl cofilinS3A-his, 1.5 μg/μl caLimk1 (k1)-myc. The extent of axon outgrowth was quantified by determining the percentage of GFP⁺, Myc⁺ or His⁺ commissural axons that had crossed any of four crossing points in their trajectory: MD, INT, MV, and FP (FIG. 2J). At low levels of GFP expression, the trajectories of individual axons can be distinguished easily at the INT, MV and FP lines. However, the extent of axonal fasciculation at the MD line makes it possible that the MD percentage is an under-representation of the reported result. The number of electroporated commissural neurons was determined by counting the number of Lhx2/9⁺ nuclei with GFP⁺ cell bodies.

CMV enhancer expression constructs were generated by inserting a Myc-tagged full-length BmprII, BmprIIΔLim-GFP or BmprIIΔLim-YFP into pcDNA3 (Invitrogen). The full-length cofilin/Limk1 and BmprIb CMV expression vectors were kind gifts of Pico Caroni and Kohei Miyazono respectively. These constructs were introduced into COS-7 cells using Lipofectamine (Invitrogen) transfection and the cell permitted to recover as previously described (Butler and Dodd, 2003). 24 hours after transfection, the COS cells were harvested in serum free Opti-MEM (Invitrogen) medium, stimulated for 30 minutes with 1 ng/ml of recombinant BMP7 protein (R&D systems) and dissociated with trypsin-EDTA (Invitrogen). The cell pellets were lysed, run on reducing SDS gels and transferred to Westem blots, which were probed with anti-p-cofilin antibodies (1:500) and anti-actin antibodies (1:2500). Western blots were developed with Supersignal West Femto Maximum Sensitivity substrate (Pierce) and the net pixel intensity of individual bands measured by densitometry using an Alpha Innotech ChemiImager 4400.

### Generation and Analysis of Mutant Mice

Limk1 embryos were genotyped by PCR (Meng et al., 2002). To assess the level of phosphorylated cofilin in litters from Limk1_{+/-} parents, spinal cords were dissected from E11.5 embryos, lysed and subjected to a Western analysis as above.

### Live Imaging of Electroporated Tissue:

Stage HH 15 chicken embryos were electroporated using 0.2 μg/μl Math1::fGFP or 0.5 μg/μl Math1::BmprIIΔLim-GFP and incubated at 37° C. until they reached stage HH19. Without removing the surrounding mesodermal tissue, the spinal cord was dissected into left and right halves and the non-electroporated side was discarded. The GFP⁺ half of the spinal cord was mounted lumen side down onto a very thin layer of type I collagen (BD Biosciences) in glass-bottom tissue culture dish (MatTek) and a further layer of collagen added to immobilize the explant. The explant was cultured in a solution of Opti-MEM and 1× pen/strep/glu (Invitrogen) and the dish was kept at 37° C. throughout the experiment using a circulating water bath. Images were taken on every 5 minutes for 6 to 8 hours, with manual refocusing when necessary, using Axiovision software on a Zeiss Axiovert 200M.

### Dissociated Cell Culture

Rat: For immunohistochemistry, cultures of dissociated E13 rat commissural neurons (Augsburger et al., 1999), were exposed to a 100 ng/ml solution of BMP7 recombinant protein for 5 minutes, fixed as previously described (Augsburger et al., 1999), and then labeled. For the Western analysis, dorsal halves of E12 rat spinal cords were dissociated using trypsin-EDTA (Invitrogen) for 5 minutes at 37° C. and the resulting neurons plated and cultured over night at 37° C. in OptiMEM. These cultures were stimulated by a 6.25 ng/ml solution of BMP7 recombinant protein diluted in OptiMEM and then analyzed by Western blotting as described above.

Chick: Chick embryos were electroporated at HH stage 14-15 with either Math1: BmprIIΔLim-GFP or CMV::BmprIIΔLim-YFP. GFP⁺ electroporated tissue was collected 24-48 hours days later (HH stage 19-24). The embryonic spinal cord was dissected into either a dorsal ⅓^{rd} or an intermediate/ventral ⅔^{rd}, in L-15 medium (Invitrogen). The neurons within these regions were dissociated using trypsin-EDTA (Invitrogen) for 5 minutes at 37° C. The neurons were resuspended in Ham's F-12 (Invitrogen) medium with L-Glutamine, Penicillin-Streptomycin-Glutamine (Invitrogen) and MITO plus Serum Extender (BD). The neurons were then plated on UV-treated glass coverslips, immunolabeled and imaged. The length of the axons and area and perimeter of each growth cone were measured using NIH Image.

### Generation and Analysis of Whole Mount Preparations

Chick embryos were in ovo electroporated at HH stage 15 and dissected at HH stage 25 without dispase treatment to remove the spinal cord from the surrounding mesoderm. The resulting explant was cut along the dorsal midline, embedded in collagen and then immediately fixed and stained with specific antisera. To quantify the number of ipsilaterally projecting GFP⁺ axons, the total number of GFP⁺ axons that had extended to the MN column was scored for the number of GFP⁺ axons that turn ipsilaterally. To quantify the directionality of the contralaterally projecting axons, the total number of GFP axons present in the RP, both those that had crossed and those in the process of crossing, were scored for the number of GFP⁺ axons that turned either rostrally or caudally. In each case, the number of ipsilaterally or contralaterally projecting axons was expressed as a percentage of the relevant total number of GFP axons.

### 1. Limk1 is Present and Active in Commissural Neurons

Canonical Bmpr signaling can directly activate Limk1 in vitro; Limk1 is thought to bind to the tail of the type II Bmpr (BmprII) in a “primed”, but inactive state (FIG. 8A). Upon BMP binding, Limk1 is phosphorylated and released into the cytosol in an activated form where it phosphorylates, and thereby inactivates, cofilin. Previous studies have suggested that Limk1 and coffin are present ubiquitously in neurons. It was confirmed that both Limk1 and its binding partner, BmprII, are present in developing commissural neurons as they extend axons, In situ hybridization and immunohistochemistry experiments on E10.5 mouse embryos demonstrated that Limk1 is expressed broadly in post-mitotic neurons and their processes in the developing spinal cord (FIG. 1A-C). In cultures of dissociated rat commissural neurons, Limk1 protein is present throughout the cell body, axon and growth cone (FIG. 1D). Strikingly, BmprII is highly enriched in commissural growth cones (arrowhead, FIG. 1E).

This localization pattern appears to have functional relevance; first antibodies against phosphorylated (p) cofilin label post-mitotic neurons in the dorsal and intermediate spinal cord (FIG. 1F) in a pattern that correlates well with the presence of Limk1 protein (FIG. 1B). p-cofilin is most dramatically expressed in the soma of these neurons however there is also faint expression in processes (arrowheads, FIG. 1F). In particular, Tag1⁺ commissural neurons are labeled (FIG. 1G), suggesting that Limk1 is actively regulating cofilin in these cells, Second, the activation status of cofilin can be regulated in dissociated commissural neurons by the brief application of BMP7 in vitro. In control, unstimulated cultures, p-cofilin was evenly distributed at low levels throughout commissural growth cones (FIG. 2A, B). In contrast, p-cofilin was upregulated within live minutes in the BMP7-stimulated growth cones (FIG. 2C, D). Importantly, the overall level of cofilin protein was not significantly different between unstimulated (FIG. 2E) and BMP7-stimulated (FIG. 2F) cultures, suggesting that BMP signaling results in the rapid phosphorylation of cofilin, rather than a redistribution of the protein. Thus, the ability of BMP7 to upregulate p-cofilin temporally precedes BMP7-mediated growth cone collapse which is observed by 20-30 minutes, suggesting that collapse could be a biological consequence of cofilin inactivation.

### 2. Constitutively Activating Limk1 Resulted in Stalled Commissural Axon Outgrowth

To determine whether Limk1 can act as an intracellular intermediate translating BMP signaling into the growth of commissural axons away from the RP, the effect of modulating Limk1 activity on commissural axon growth was assessed in vivo. Inventors first expressed a constitutively active Myc-tagged form of Limk1 (caLimk1-myc) within chick commissural axons by in ovo electroporation. Constructs were generated containing either farnesylated (f) GFP or caLimk1-myc-IRES-fGFP under the control of the Math1 enhancer, which directs the expression of genes to commissural neuron progenitors. In control experiments, GFP⁺ commissural neurons projected axons normally across the FP by Hamilton Hamburger (HH) stage 23 (FIG. 3A, B). In contrast, commissural neurons electroporated with the Math1::caLimk1-myc-IRES-fGFP construct displayed severe defects in axon extension (FIG. 3C-E), with GFP⁺ growth cones observed only immediately adjacent to the cell bodies (arrowhead, FIG. 3E). The extent of outgrowth was quantified by determining whether commissural axons had crossed one of four arbitrarily drawn lines in the spinal cord: mid-dorsal (MD), intermediate (INT) or mid-ventral (MV) spinal cord or the FP (FIG. 3H). By HH stage 23, 67% of control GFP⁺ axons have projected to the intermediate spinal cord, whereas only 8% of Myc⁺ axons extend this far (FIG. 3I). This defect did not result from an alteration in commissural neural fate, similar numbers of Lhx2/9⁺ (Lh2a/b) neurons were observed on the electroporated and non-electroporated sides in both control and experimental conditions.

These results indicate that Limk1 activation can inhibit commissural axon outgrowth by phosphorylating cofilin and thereby freezing actin dynamics in the commissural growth cone. Consistent with this model, the stall in axon growth could be rescued by the concomitant electroporation of cofilinS3A-his, a His-tagged non-phosphorylatable form of cofilin, in commissural neurons (FIG. 3F, G). Axon outgrowth was partially restored, with 47% of His* Myc⁺ axons now reaching the INT line (FIG. 3I). In contrast, the concomitant electroporation of a wild type form of cofilin did not rescue the caLimk1 phenotype,

### 3. Lowering Limk1 Activity Accelerates Commissural Axon Outgrowth

To assess the effect of lowering Limk1 activity on the commissural axon trajectory, the inventors first analyzed E10.5 Limk1 mutant mice (Meng et al., 2002) using a Math1:tauGFP reporter (Imondi et al., 2007) to detect the population of commissural axons that arises from Math1 neural progenitors. The level of phosphorylated cofilin was decreased in Limk1^{-/-} spinal cords (FIG. 9), suggesting that cofilin is more active in these mice. In the transverse plane of the spinal cord, commissural axons first extend circumferentially away from the dorsal midline (Augsburger et al., 1999), and then project towards the FP at the ventral midline (Tessier-Lavigne et al., 1988; Placzek et al., 1990). The individual trajectories of GFP+ axons could be distinguished only at the earliest stages of commissural axon circuit formation. Although the orientation of Limk1^{-/-} axons away from the RP was indistinguishable from wild-type littermates (FIG. 4A, D), the extent of axon growth in Limk1^{-/-} spinal cords appeared to be more advanced than in the controls (Towheads, FIG. 4B, E). This phenotype could only be unambiguously quantified by examining the number of GFP+ commissural axons crossing the FP. At the earliest stages, the spinal cords from the Limk1^{-/-} embryos had up to 25% more axons present in the FP compared to wild-type littermates (FIG. 4C, F, G), suggesting that reducing the level of Limk1 activity accelerates the growth of pioneering Math1⁺ commissural axons.

To determine whether BMP signaling regulates the activity of Limk1. In commissural neurons, the inventors sought to decrease the level of Limk1 activity in a BMP dependent manner in vivo. To this end, the inventors generated a truncated version of BmprII in which the Limk1 binding site on the intracellular tail of BmprII was replaced with GFP (BmprIIΔLim-GFP). It was thought that this construct would compete with the endogenous chick BmprII for activation by the RP BMPs, thereby resulting in the sequestration of Limk1 (FIG. 8B). Supporting this model, overexpressing BmprIIΔLim-GFP in COS cells resulted in a significant decrease in coffin phosphorylation (FIG. 8C, D), suggesting that the truncated form of BmprII acts to lower Limk1 activity. To assess whether reducing Limk1 activity in a BmprII-dependent manner affects commissural axon outgrowth in vivo, chick embryos were electroporated with either Math1::BmprIIΔLim-GFP or Math1::fGFP constructs and permitted to develop until stage 19-21. At these stages, control GFP⁺ axons are in the process of projecting towards the ventral midline; for example, at HH stage 20 the Math1⁺ population of commissural axons have reached the intermediate spinal cord (arrowheads. FIG. 5B, F) but have not yet approached the FP. In striking contrast, commissural axons expressing Math1::BmprIIΔLim-GFP have progressed much further by the same stages, in many cases reaching and crossing the FP (arrowheads, FIG. 5D, H). The inventors quantified this behavior for HH stage 19-21 using the scheme described in FIG. 3H. At each of these stages, the control and experimental commissural neurons initially extended similar numbers of axons, however in all cases the BmprIIΔLim-GFP⁺ axons had projected further (FIG. 5I). At HH stage 20, for example, less than 2% of the control population of axons had reached the FP, compared to over 25% of the experimental population of neurons (FIG. 5I). Accelerated axon growth was only observed when the truncated form of BmprII could be activated by BMPs. Introducing a form of BmprII in which both the extracellular domain (E) and Lim binding domain had been deleted (BmprIIΔEΔLim-GFP) into Math1⁺ neurons had no observable effect on the rate of axon growth (FIGS. 5J and 10). Thus, the ability of BmprIIΔLim-GFP to downregulate Limk1 does depend on the endogenous BMP signals from the RP.

Accelerated commissural axon outgrowth was not a consequence of premature or altered commissural neural development. The activity of canonical effectors of the BMPs, the Smads, was unaffected by the electroporation of either GFP or the experimental constructs (FIG. 11) and there was no significant difference between the timing and number of Lhx2/9⁺ born in control and experimental spinal cords (FIG. 12). Rather, the ability of axons to grow faster appears to be a consequence of increased cofilin activity. The overexpression of cofilin in commissural neurons in vivo also resulted in accelerated axon growth to the FP (FIG. 13). Axons expressing high levels of cofilin grew with very similar kinetics to the axons misexpressing BmprIIΔLim-GFP (FIG. 13G).

Taken together, these data suggest that elevating cofilin activity, either directly or by reducing Limk1 activation in a BMP-dependent manner, results in commissural axons growing faster and thereby reaching their intermediate target approximately a day earlier than control axons. Strongly supporting this model, imaging live axons in explants of electroporated chick tissue in vitro demonstrated that the BmprIIΔLim-GFP⁺ axons had a 30% faster average velocity than control GFP axons (FIG. 5K) as they grew through the dorsal spinal cord. This increased rate of growth would permit them to grow an average of 100 μm further than control axons in a 24-hour period, a figure strikingly consistent with the changes in axon length observed after electroporating the spinal cord with BmprIIΔLim-GFP (FIG. 5I). Interestingly, the growth rate of GFP⁺ and BmprIIΔLim-GFP⁺ axons was different only in the dorsal spinal cord; there was no significant difference (p>0.26) between the growth rate of these two populations of axons in the ventral spinal cord (FIG. 5K). The velocity of control GFP⁺ axons was 30% faster in the ventral spinal cord whereas BmprIIΔLim-GFP+ axons projected through the ventral spinal cord at a comparable rate to that in the dorsal cord (FIG. 5K). Taken together, these results demonstrate Math1⁺ commissural axons normally extend more slowly in the dorsal spinal cord than in the ventral spinal cord. Reducing the activity of Limk1 appears to release commissural axons from this inhibition of growth, such they now grow at a constant “accelerated” speed through the spinal cord.

### 4. Lowering Limk1 Activity Results in Long Filopodial Protrusions

Activated cofilin depolymerizes actin filaments and thereby increases the pool of actin monomers needed at the leading edge of a cell for motility. Thus, the cellular basis for accelerated commissural axon growth is likely to be an increase in the rate of actin polymerization. The inventors assessed this possibility by determining whether the manipulations of cofilin activity altered the morphology of the actin cytoskeleton in growth cones. Following the electroporation of either cofilin or BmprIIΔLim-GFP, dorsal spinal neurons were observed to extend growth cones with longer, more complex filopodia both in vivo (FIG. 6B) and in vitro (FIG. 6E, F) than control growth cones (FIG. 6A, C, D). No difference in overall neurite length was observed in vitro (FIG. 6G), presumably because the neurons are no longer adjacent to a source of BMPs. However, the perimeter of the experimental dissociated growth cones was 40% longer than that of control growth cones (FIG. 6G). Increased protrusions from the leading edge are consistent with actin polymerization being more dynamic in the growth cone.

Taken together, these observations support the model that the balance between the activation states of cofilin and Limk1 controls rate of axon outgrowth in vivo by regulating how fast actin treadmills in the growth cone. This mechanism may be a general one: both Limk1 and cofilin are widely expressed in neurons (see also FIG. 1A, B) and dissociated intermediate and ventral spinal neurons misexpressinu BmprIIΔLim-YFP also have more complex growth cones (FIG. 14).

### 5. Accelerated Commissural Neurons Make Guidance Errors Projecting Towards and Across the FP

These studies indicate that, in addition to polarizing commissural axons, a key role of the BMP chemorepellent in vivo is to regulate the rate at which commissural axon extend away from the dorsal midline. The existence of such an axon outgrowth-regulating activity would permit commissural axons to reach an intermediate targets, such as the FP, at a particular time or speed in development. It was then assessed whether accelerated axon outgrowth affected the ability of commissural axons to make the correct guidance decisions in the ventral spinal cord.

The trajectory of axons originating from Math1⁺ neurons was visualized in longitudinal “open book” preparations of the spinal cord. Control GFP⁺ chick axons (FIG. 7A, B) behaved similarly to what has been previously described for the analogous class of rodent axons as follows: by HH stage 24, there were two populations of axons, the well-described commissural axons that project contralaterally, first across the FP then rostrally towards the brain (solid bracket, FIG. 7A arrowhead, FIG. 7B) and another, later born class of axons that project ipsilaterally, turning rostrally before crossing the FP (dotted bracket, FIG. 7A, arrow, FIG. 7B). This subdivision of Math1 neurons arises from spatial and temporal segregation of the Lhx9 transcription factor. Lhx9⁺ neurons were born normally following electroporation with the Math1::BmprIIΔLim-GFP construct and project in a polarized manner around the circumference of the spinal cord (FIG. 9). However, their axons displayed guidance errors on reaching the ventral spinal cord. Very few of the accelerated GET axons were observed to turn ipsilaterally (FIG. 7C-E) and although many of the accelerated commissural axons still projected rostrally about 10% of the axons inappropriately turned caudally (arrowheads, FIG. 7C, D, F). Very similar guidance errors were observed when Math1⁺ axons were electroporated with the cofilin-myc construct (FIG. 7E, F), suggesting that these errors are a consequence of modulating the status of cofilin activity.

Thus, elevating the activation state of cofilin has a profound consequence for the two populations of Math1⁺ axons, they ignore and/or misinterpret guidance signals, such that they make significant turning errors.

### Example 2

This example shows that, in addition to commissural axons, modulation of the Limk1/cofilin balance also has profound effect on the growth of spinal motor axons.

### 1. Both Limk1 and Cofilin are Present in Motor Neurons

The ability of Limk1/cofilin to regulate axon outgrowth is likely to be a general property of neurons. It is shown here that cofilin is present ubiquitously in neurons and Limk1 is expressed many post-mitotic neurons in the spinal cord (FIG. 15A). In particular, Limk1 is expressed in spinal motor neurons (MNs) as they extend axons into the periphery (FIG. 15A). Limk1 appears to be active in these neurons given that expression of Limk1 coincides with high levels of phosphorylated cofilin (FIG. 15B). Thus, it is feasible that Limk1/cofilin regulate the rate of motor axon outgrowth to shape the trajectory of motor axons.

### 2. Loss of Limk1 Results in Overgrowth Defects in the Embryonic Limb, Suggestive of Accelerated Growth

Mice mutant for Limk1 has been studied for abnormalities in the formation of motor circuits. Starting from developmental stage E9.5, LMC motor axons start to extend out of the ventral horn of the spinal cord towards the developing limbs. Motor axons innervating the limb initially project their axons along a common path, but at the base of the limb, the motor nerve bifurcates at the plexus to form distinct dorsal and ventral branches. These results indicate that motor axons deficient for Limk1 make guidance errors as they innervate the limb (FIG. 16). Limk1^{-/-} axons appear to branch inappropriately at the plexus (arrows, FIGS. 16B and 16D), the dorsal branch extends further into the limb compared to controls (open arrowhead, FIGS. 16B and 16D) and the ventral branch appears to be defacisculated as it extends into the limb mesenchyme (arrowheads, FIGS. 16B and 16D). These phenotypes are consistent with those seen for Limk1^{-/-} commissural axons (Example 1) and suggest that, as in commissural axons, the loss of Limk1 results in accelerated motor axonal growth.

3. Upregulation of Cofilin in Motor Neurons Results in their Extending Faster Growing Motor Axon In Vivo

It's been observed that overexpressing cofilin in MNs in vivo results in their more rapidly innervating the embryonic limb. Cofilin-myc (CMV::cofilin-myc) was misexpressed throughout one side of the chicken embryonic spinal cord using in ovo electroporation and the effect on motor axon growth was monitored by electroporating cherry into motor neurons on both sides of the spinal cord under the control of the Hb9 enhancer (Hb9::cherry). By Hamilton Hamburger (HH) stage 24/25, both the control and experimental motor axons have reached the plexus (arrows, FIG. 17B). However, whereas most of the control motor axons have paused at the plexus, many of the experimental cofilinmyc+ motor axons have extended significantly further into the limb (arrowhead, FIG. 17B). Thus overexpression of cofilin in motor axons appears to accelerate their growth as was seen for spinal commissural axons (Example 1).

4. Upregulation of Cofilin in Motor Neurons Results in their Extending Significantly Larger and More Complex Growth Cones In Vitro

It has been further examined whether increasing cofilin activity alters the morphology of the actin cytoskeleton in motor axon growth cones. In a similar result to that observed for commissural axons (Example 1), after the electroporation of either cofilin or BmprIIΔLim-YFP, dissociated spinal MNs extend growth cones with longer, more complex filopodia in vitro (FIG. 18D-G) than control growth cones (FIG. 18A-C). The average perimeter of these growth cones is almost 50% longer (p<2.2×10-5, student's t-test) than the perimeter of control growth cones. Such increased protrusions from the leading edge are generally observed after upregulating the activity of cofilin and are consistent with actin polymerization being more dynamic in the growth cone. Taken together, these results indicate that overexpressing cofilin in motor axons has the same biological effect that it is shown in commissural axons. This is strong evidence that the overexpression of cofilin in neurons is a mechanism that can be used to generally accelerate axon outgrowth.

### Example 3

This example examines the effect of introducing cofilin into different classes of embryonic stem (ES)-cell derived neurons

1. Deriving Neurons from Stem Cells

Motor neurons: MNs were made using a well-established protocol in which ES cells are first permitted to form embryonic bodies (EBs) and are then treated with a combination of retinoic acid and solubilized sonic hedgehog protein. This protocol results in the robust induction of both Olig2⁺ MN progenitors and Isl1/2⁺ MNs within EBs after 5 days in culture (FIG. 19A).

For commissural neurons, the combination of growth factors has been determined that allows to derive a class of sensory interneurons that relays sensory information (temperature, pain etc) to the brain from mouse ES cells. By adding retinoic acid and Bone Morphogenetic Protein (BMP) 4 to EBs, Math1 dl1 spinal commissural neurons were generated for the first time after 7-9 days in culture (FIGS. 19B and C).

2. Introducing Cofilin into ES-Cell Derived Neurons

Introduction of cofilin into ES-cell derived neurons are carried with the following procedure. Transient transfection: it has been found that mouse ES cells can be successfully transfected with either CMV::GFP (FIGS. 20A and B), CMV::cofilin-myc (FIGS. 20D and E) or control (FIG. 20C) with lipofectamine.

Stable integration is carried out with a replication incompetent Lentiviral vector (based on pLenti), that will result in cofilin being integrated and thereby stably over-expressed in ES-cell derived neurons after the differentiation procedure.

Unless otherwise defined, all technical and scientific terms used herein have the same meaning as commonly understood by one of ordinary skill in the art to which this invention belong. All nucleotide sequences provided herein are presented in the 5′ to 3′ direction.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control, REFERENCES
Arber S, Barbayannis F A, Hanser H, Schneider C, Stanyon C A, Bernard O, Caroni P (1998) Regulation of actin dynamics through phosphorylation of coffin by LIM-kinase. Nature 393:805-809.
Augsburger A, Schuchardt A, Hoskins S. Dodd J, Butler S (1999) BMPs as mediators of roof plate repulsion of commissural neurons. Neuron 24:127-141.
Birgbauer E, Solomon F (1989) A marginal band-associated protein has properties of both microtubule- and microfilament-associated proteins. J Cell Biol 109:1609-1620.
Butler S J, Dodd J (2003) A role for BMP heterodimers in roof plate-mediated repulsion of commissural axons. Neuron 38:389-401,
Evan G I, Lewis G K, Ramsay G, Bishop J M (1985) Isolation of monoclonal antibodies specific for human c-myc proto-oncogene product. Mol Cell Biol 5:3610-3616.
Geisert E E, Jr., Frankfurter A (1989) The neuronal response to injury as visualized by immunostaining of class III beta-tubulin in the rat. Neurosci Lett 102:137-141.
Lee K J, Mendelsohn M, Jessell T M (1998) Neuronal patterning by BMPs: a requirement for GDF7 in the generation of a discrete class of commissural interneurons in the mouse spinal cord. Genes Dev 12:3394-3407.
Liem K F, Tremml G, Jessell T M (1997) A role for the roof plate and its resident TGFbeta-related proteins in neuronal patterning in the dorsal spinal cord. Cell 91:127-138.
Meng Y, Zhang Y, Tregoubov V. Janus C, Cruz L, Jackson M, Lu W Y, MacDonald J F, Wang J Y, Falls D L, Jia Z (2002) Abnormal spine morphology and enhanced LIP in LIMK-1 knockout mice. Neuron 35:121-111.
Rosenzweig B L, Imamura T, Okadome T, Cox ON, Yamashita H, ten Dijke P, Heldin C H, Miyazono K (1995) Cloning and characterization of a human type II receptor for bone morphogenetic proteins. Proc Natl Acad Sci USA 92:7632-7636.
Ruegg M A, Stoeckli E T, Kuhn T B, Heller M, Zuellig R. Sonderegger P (1989) Purification of axonin-1, a protein that is secreted from axons during neurogenesis. Embo J 8:55-63.
Yamauchi K, Phan K D, Butler S J (2008) BMP type I receptor complexes have distinct activities mediating cell fate and axon guidance decisions. Development 135:1119-1128.

What is claimed is:

1. A method for promoting extension of a neural cell, comprising increasing the biological activity of cofilin in the cell, thereby promoting extension of the neural cell.

2. The method of claim 1, wherein the increasing of the biological activity of cofilin in the cell comprises introducing into the cell an isolated or recombinant cofilin polypeptide or an isolated or recombinant polynucleotide encoding the polypeptide.

3. The method of claim 2, wherein the isolated or recombinant cofilin polypeptide comprises a mutation that inhibits phosphorylation of the cofilin polypeptide.

4. The method of claim 3, wherein the mutant cofilin polypeptide comprises the amino acid sequence of SEQ ID NO: 1 or a biological equivalent thereof.

5. The method of claim 1, wherein the increasing of the biological activity of cofilin in the cell comprises inhibiting the expression or the biological activity of Limk1 in the cell.

6. The method of claim 5, wherein the inhibiting of the activity of Limk1 in the cell comprises introducing into the cell an isolated or recombinant Limk1 polypeptide mutant that does not phosphorylate cofilin or an isolated or recombinant polynucleotide encoding the polypeptide mutant.

7. The method of claim 6, wherein the Limk1 polypeptide mutant does not have one or more of a LIM or a PDZ domain.

8. The method of claim 1, wherein the increasing of the biological activity of cofilin in the cell comprises inhibiting the expression or the biological activity of BmprII.

9. The method of claim 8, wherein the inhibiting of the biological activity of BmprII comprises introducing into the cell an isolated or recombinant BmprII polynucleotide mutant that does not phosphorylate Limk1 or an isolated or recombinant polynucleotide encoding the BmprII polynucleotide mutant.

10. The method of claim 9, wherein the BmprII polynucleotide mutant comprises the amino acid sequence of SEQ ID NO: 2 or a biological equivalent thereof.

11. The method of claim 1, wherein the biological activity of cofilin in the neural cell is increased at a location in the cell proximate to an end of the cell in need of extension.

12. The method of claim 1, wherein the neural cell is a neural stem cell or a neural precursor cell.

13. The method of claim 12, wherein the neural stem cell is derived from an induced pluripotent stem cell (iPSC), an embryonic stem cell or a parthenogenetic stem cell.

14. The method of claim 1, wherein the neural cell is a damaged or degenerated neural cell that is terminally differentiated.

15. The method of claim 1, wherein the increasing of the biological activity of cofilin is in vivo or ex vivo.

16. The method of claim 1, wherein the neural cell is a human neural cell.

17. The method of claim 1, wherein the neural cell comprises a commissural axon or a motor axon.

18. An extended neural cell prepared by a method of claim 1.

19. A neural cell comprising an isolated or recombinant polypeptide comprising an amino acid sequence of SEQ ID NO: 1 or 2 or a biological equivalent thereof, or an isolated or recombinant polynucleotide comprising a nucleic acid sequence encoding SEQ ID NO: 1 or 2 or a biological equivalent thereof.

20. The neural cell of claim 19, wherein the polypeptide or polynucleotide is localized at a location in the cell proximate to an end of the cell in need of extension.

21. The neural cell of claim 19, wherein the neural cell is a neural stem cell or a neural precursor cell.

22. The neural cell of claim 21, wherein the neural stem cell is derived from an induced pluripotent stem cell (iPSC), an embryonic stem cell or a parthenogenetic stem cell.

23. The neural cell of claim 19, wherein the neural cell is a damaged or degenerated neural cell that is terminally differentiated.

24. A population of neural cell of claim 19.

25. A method for treating a neurological disease characterized by a damaged or a degenerated neural cell, comprising increasing the biological activity of cofilin in the neural cell to promote the extension of the neural cell, thereby treating the disease.

26. The method of any claim 25, wherein the neurological disease is selected from Traumatic Brain injury, Alzheimer's disease, Parkinson's disease, epilepsy, Huntington's disease or stroke.

27. A method for treating a neurological disease characterized by a damaged or a degenerated neural cell, comprising introducing to the neural cell an isolated or recombinant polypeptide comprising an amino acid sequence of SEQ ID NO: 1 or 2 or biological equivalent thereof, or an isolated or recombinant polynucleotide comprising a nucleic acid sequence encoding SEQ ID NO: 1 or 2 or a biological equivalent thereof.

28. A method of identifying an agent suitable for increasing the biological activity of cofilin, comprising contacting a candidate agent with a neural cell, wherein increased extension of the neural cell and increased phosphorylation of cofilin as compared to a neural cell not in contact with the agent indicates that the agent is suitable for increasing the biological activity of cofilin.


SEQUENCE LISTING (6 sequences)

SEQ ID NO: 1 - protein, 166 aa, Rattus norvegicus
MAAGVAVSDGVIKVFNDMKVRKSSTPEEVKKRKKAVLFCLSEDKKNIILEEGKEILVGDVGQTVDDPYTTFVKMLPDKDCRYALYDATYETKESKKEDLVFIFWAPESAPLKSKMIYASSKDAIKKKLTGIKHELQANCYEEVKDRCTLAEKLGGSAVISLEGKPL

SEQ ID NO: 2 - protein, 529 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNER

SEQ ID NO: 3 - DNA, 20 bp, Artificial Sequence
  Description of Artificial Sequence Synthetic primer
agggatctgaatccccaaac

SEQ ID NO: 4 - DNA, 43 bp, Artificial Sequence
  Description of Artificial Sequence Synthetic primer
gagattaaccctcactaaagggaacaatcccatccccctaaac

SEQ ID NO: 5 - DNA, 31 bp, Artificial Sequence
  Description of Artificial Sequence Synthetic primer
gccgccacatgtcttcctcgctgcagcggcc

SEQ ID NO: 6 - DNA, 34 bp, Artificial Sequence
  Description of Artificial Sequence Synthetic primer
gccgccactagtgacaggttgcgttcattctgca